(19) United States
(12) Patent Application Publication
(10) Pub. No.: US 2005/0129731 A1
(43) Pub. Date: Jun. 16, 2005

(54) Biocompatible, biostable coating of medical surfaces

(76) Inventors: Roland Horres, Stolberg (DE); Michael Hoffmann, Eschweiler (DE); Volker Faust, Aachen (DE); Erika Hoffmann, Eschweiler (DE); Donato Di Biase, Aachen (DE)

Correspondence Address: AMIN & TUROCY, LLP, 1900 EAST 9TH STREET, NATIONAL CITY CENTER, 24TH FLOOR,, CLEVELAND, OH, 44114, US

(21) Appl. No.: 10/979,977

(22) Filed: Nov. 3, 2004

Related U.S. Application Data
(60) Provisional application No. 60/516,295, filed on Nov. 3, 2003.
(60) Provisional application No. 60/571,582, filed on May 17, 2004.

(51) Int. Cl. A61F 2/06; A61K 38/16; A61K 31/704; A61K 31/737; A61K 31/4745; A61K 31/365; A61K 35/78

(52) U.S. Cl. 424/423; 623/1.42; 514/27; 514/28; 514/183; 514/12; 514/33; 514/339; 514/291; 424/731; 514/169; 514/54; 514/460; 514/548; 514/283; 514/449

(57) ABSTRACT

The invention relates to medical products with at least one biocompatible biostable polysulfone coating with which the elution kinetics of the incorporated and/or deposited at least one antiproliferative, antiinflammatory, antiphlogistic and/or antithrombotic active agent can be controlled via the admixing of at least one hydrophilic polymer in a suitable amount and as well as an local separation of different active agents and active agent combinations respectively can be achieved by means of the layer system of biostable polymers, methods of manufacturing these medical products as well as their use especially in the form of stents for prevention of restenosis.

### RELATED APPLICATIONS

This application claims priority under 35 U.S.C. § 119(e) to U.S. Provisional Patent Application No. 60/516,295 filed on Nov. 3, 2003 and to U.S. Provisional Patent Application No. 60/571,582 filed on May 17, 2004, both of which are hereby incorporated by reference.

The invention relates to medical surfaces with a biocompatible, biostable coating of polysulfones and/or polysulfone derivatives resp. copolymers with polysulfone containing and/or covered by at least one antiproliferative, antiinflammatory, antiphlogistic and/or antithrombotic active agent, methods for manufacturing of these surfaces as well as their use in form of long-term implants, especially stents for the prevention of restenosis.

The implantation of stents using balloon dilatation of occluded vessels increasingly established in the last years. Although stents decrease the risk of a renewed vessel occlusion they are until now not capable of preventing such restenoses completely.

An exact conceptual description of restenosis cannot be found in the technical literature. The most commonly used morphologic definition of the restenosis is the one which defines the restenosis after a successful PTA (percutaneous transluminal angioplasty) as a reduction of the vessel diameter to less than 50% of the normal one. This is an empirically defined value of which the hemodynamic relevance and its relation to clinical pathology lacks of a massive scientific basis. In practical experience the clinical aggravation of a patient is often viewed as a sign for a restenosis of the formerly treated vessel segment.

There are three different reasons for the restenosis caused by the stent:
a.) During the first period after the implantation the stent surface is in direct contact with the blood and an acute thrombosis can occur which again occludes the vessel due to the now present foreign surface.
b.) The implantation of the stent generates vessel injuries which also induce inflammation reactions which play an important role for the recovery process during the first seven days in addition to the above mentioned thrombosis. The herein concurrent processes are among others connected with the release of growth factors which initiate an increased proliferation of the smooth muscle cells which rapidly leads to a renewed occlusion of the vessel, because of uncontrolled growth.
c.) After a couple of weeks the stent starts to grow into the tissue of the blood vessel. This means that the stent is surrounded totally by smooth muscle cells and has no contact to the blood. This cicatrization can be too distinctive (neointima hyperplasia) and may lead to not only a coverage of the stent surface but to the occlusion of the total interior space of the stent.

It was tried vainly to solve the problem of restenosis by the coating of the stents with heparin (J. Whörle et al., European Heart Journal (2001) 22, 1808-1816). Heparin addresses as anti coagulant only the first mentioned cause and is moreover able to unfold its total effect only in solution. This first problem is meanwhile almost totally avoidable medicamentously by administration of anti-coagulants. The second and third problem is intended now to be solved by inhibiting the growth of the smooth muscle cells locally on the stent. This is carried out by e.g. radioactive stents or stents which contain pharmaceutically active agents.

U.S. Pat. No. 5,891,108 discloses for example a hollow moulded stent, which can contain pharmaceutical active agents in its interior, that can be released throughout a various number of outlets in the stent. Whereas EP-A-1 127 582 describes a stent that shows ditches of 0.1-1 mm depth and 7-15 mm length on its surface which are suitable for the implementation of an active agent. These active agent reservoirs release similarly to the outlets in the hollow stent the contained pharmaceutically active agent in a punctually high concentration and over a relatively long period of time which however leads to the fact that the smooth muscle cells are not anymore or only very delayed capable of enclosing the stent. As a consequence the stent is much longer exposed to the blood, what leads again to increased vessel occlusions by thromboses (Liistro F., Colombo A., Late acute thrombosis after Paclitaxel eluting stent implantation. Heart (2001) 86, 262-4).

One approach to this problem is represented by the phosphorylcholine coating of biocompatibles (WO 0101957), as here phosphorylcholine, a component of the erythrocyte cell membrane, shall create a non thrombogeneous surface as a component of the deposited non biodegradable polymer layer on the stent. Dependent of its molecular weight, thereby the active agent is absorbed by the polymer containing phosphorylcholine layer or adsorbed on the surface.

Object of the present invention is, to provide a medical product with a hemocompatible surface as well as a method of manufacturing this medical product with the hemocompatible surface.

Especially the hemocompatible surface of the medical product shall allow a continuous and controlled ingrowth of the medical product into the vessel wall.

This object is solved by the technical teaching of the independent claims of the present invention. Further advantageous embodiments of the invention are evident from the dependent claims, the description as well as the examples.

The present invention relates to medical products the surface(s) of which is(are) at least partially coated with at least one biostable polysulfone layer.

It was surprisingly found that the coating of medical surfaces being in permanent contact with blood, with polysulfone, polyethersulfone and/or polyphenylsulfone and its derivatives represents an extremely well suitable biocompatible carrier for active agents. By admixing of hydrophilic biocompatible polymers or by using polysulfones with ambivalent properties, i.e. with lipophilic and hydrophilic groups the pore size of the polysulfone matrix can be varied so that hereby a plurality of variations can be achieved in respect of the used active agents, their administrable amount as well as the desired release rate. Especially the elution kinetics of the at least one active agent can be regulated through the pore size in the biostable layer. The pore size is in turn determined by the type and amount of the used hydrophilic polymer resp. the amount of lipophilic and lipophobic groups in the polysulfone or polysulfone mixture. Besides the influence of the admixed hydrophilic polymer the addition of small amounts of water (or also ethyl acetate) in the coating solution influences the future properties of the active agent loaded coated implant. The setting of the load distribution, the release properties (as a function of the time and the amount of eluted active agent) and the spraying properties of the coating solution are decisively formed by the defined admixing of water (or also ethyl acetate or other additives described more below) into the spraying solution It proved also to be advantageous, that the use of nitrogen as carrier gas to the spray coating leads to a load of the active agent containing polymer layer with nitrogen, which remains in the layer and provides here for the intactness of the active agent in its property as protective gas. Therewith the shelf life of the active agent is guaranteed in unaltered active form permanently.

The modification of the polysulfone framework by polymer analogous reactions such as the preparation of new polysulfone copolymers (e.g. as polysulfone blockcopolymers or in statistical distribution) has influence on the physical performance of the resulting polymers, whereby the properties of the polymer can be controlled, and are usable whether in combination with the non-modified polymers or individually as new hemocompatible coating material. In this way a carboxylic groups containing polyethersulfone can be prepared via the reaction of polysulfone copolymers with 4,4′-bis(hydroxyphenyl)pentanoic acid (BPA), which leads to a clear hydrophilic property of the polymer. The properties of the hydrophilic polysulfone can be used also as hydrophilic polymer addition to the non-modified polysulfone as already mentioned above. Via the setting of the modification grade the hydrophilic property grade is influenced, so that a polymer molecule results, in which every chain contains non-modified and modified regions and so associates in itself hydrophobic and hydrophilic properties, which impart the polymer also an altered sterical assembly of the chain segments, the so-called secondary structure. Therefore it is preferred to use a polysulfone for the coating, which features hydrophilic regions and hydrophobic regions. Suchlike polysulfones can be prepared by providing a polysulfone with hydrophilic side chains or functional groups after the polymerization via polymer analogous reactions if the polymer itself is hydrophobic or contrary a hydrophilic polysulfone is provided with hydrophobic side chains or functional groups. In this preferred embodiment the hydrophilic and hydrophobic properties are associated in one polymer molecule, generally with a statistic distribution, as the polymer analogous reactions take place with a statistic distribution. Further such systems can be prepared from hydrophilic polysulfone with hydrophobic polysulfone via statistic polymerization of at least one hydrophilic monomer and at least one hydrophobic monomer. Hereby similar structures result such as in the afore-mentioned embodiment of the subsequent modification via polymer analogous reactions. A third embodiment consists in the blockcopolymerization of at least one hydrophilic sulfone blockcopolymer with at least one hydrophobic sulfone blockcopolymer into a polysulfone, which respectively features the hydrophilic and hydrophobic properties in the individual blocks. Another modification is to react at least one hydrophilic monomer in an alternating copolymerization with at least one hydrophobic monomer. Here the hydrophilic and hydrophobic properties are distributed alternating in the polymer chain in the obtained polysulfone. Further in the coating according to invention a mixture of at least one hydrophilic polysulfone with at least one hydrophobic polysulfone can be used. Here the hydrophilic and hydrophobic properties are not associated in one polymer molecule but can be found in the coating and result the same effects as in the previously mentioned embodiments.

For the preparation of the polysulfones are suitable all of the polymerization reactions known to the skilled in the art such as radical, anionic, cationic or thermal polymerization. Examples for the afore-mentioned polysulfones as well as possibilities for their preparation will be described more below.

Further there is the possibility to derivatize introduced functional groups such as the carboxylic group (Macrom. Chem. Phys. 1994, 195, 1709).

So e.g. via introduction of fluorinated compounds the hydrophobic property of the active agent can be increased beyond the hydrophobic properties of the used polymer (Coll. Polym. Sci. 2001, 279, 727). Via the introduction of functional groups graft copolymers can be prepared, whereas the side chains now consist of other structure units than the major chain. Thereto biocompatible, biostable and biodegradable polymers can be used.

The functional groups can be used also for a hydrolysis weak bonding of active agents. The active agent is released due to its form, which is also controlled through the hydrolysis and in dependence from the type of bonding (thioester bonding, ester bonding). Here the advantage exists in the possibility to control the elution of the active agent such that the release curve takes another trajectory and adaptations to many various courses of disease with diverse requirements to the active agent concentration in the dependence of time can be achieved with the implant. A variation is the covalent bonding of desulphated and N-reacetylated heparin and/or N-carboxymethylated and/or partially N-acetylated chitosan to the polymer chain, whereby the hemocompatibility of the polymer is improved by means of the athrombogeneous compound.

Through the possibility of the assembly of at least two layers of the polymer, which can be varied in its composition, as well as in the variation of the additives moreover a layer dependent differentiation regarding the used active agents as well as regarding the concentration can be carried out. This capability of adaptation distinguishes the polysulfone matrix as a universally usable biostable coating material for the prevention of the restenosis.

For the setting of the pore size and therewith of the active agent amount in the polysulfone matrix not only hydrophilic polymers but also materials and water itself can be used as additives. The pore size controls on the one hand the release kinetics of the at least one antiproliferative, antiinflammatory, antiphlogistic and/or antithrombotic active agent as well as in particular embodiments the amount of active agent, which can be introduced resp. deposited in a polysulfone coating, as the pores in the polysulfone can serve as an active agent reservoir.

For the generation of pores in the polysulfone matrix during the use of these additives different strategies can resp. have to be followed.

In principle the generation of pores is carried out such that the additives with the matrix building polysulfone are deposited together on the medical product to be coated according to a suitable method. Here dependent from the differences in the hydrophilic property of the used additives as well as the matrix building polysulfone homogenous compartments of the additive are formed, which can be controlled in their dimension. The number of these homogenous compartments per volume unit of the polysulfone matrix can be controlled through the percentagely added amount of the additive.

As additives can be used in detail amino acids, polyamino acids, hydrophilic polymers, saccharides, oligosaccharides, polysaccharides, oligopeptides, polyvinylpyrrolidone, polyethylenimine, glycerine, polyethers, glycol, minerals and water.

In the case of the amino acids the genetically coded acidic amino acids asparaginic acid, glutaminic acid; the neutral amino acids alanine, asparagine, cysteine, glutamine, glycine, isoleucine, leucine, methionine, phenylalanine, proline, serine, threonine, tryptophan, tyrosine, valine; and the basic amino acids arginine, histidine, lysine; as well as the genetically non coded amino acids ornithine and taurine are preferred. Especially preferred are the representatives of the L-series of these amino acids. Further the representatives of the D-series of these amino acids as well as D,L-mixtures of one amino acid as well as D,L-mixtures of more amino acids are preferred.

In the case of the polyamino acids the amino acids poly-L-asparaginic acid, poly-L-glutaminic acid, poly-L-alanine, poly-L-asparagine, poly-L-cysteine, poly-L-glutamine, poly-L-glycine, poly-L-isoleucine, poly-L-leucine, poly-L-methionine, poly-L-phenylalanine, poly-L-proline, poly-L-serine, poly-L-threonine, poly-L-tryptophan, poly-L-tyrosine, poly-L-valine, poly-L-arginine, poly-L-histidine, poly-L-lysine as well as poly-ornithine and poly-taurine are preferred. Further are also representatives of the D-series of these polyamino acids as well as D,L-mixtures of one polyamino acid as well as D,L-mixtures of more polyamino acids are suitable.

In the case of the hydrophilic polymers globular molecules such as organic nanoparticles, star polymers, dendrimers and/or highly (super) branched polymers are preferred.

In the case of the minerals carbonates, chlorates, phosphates and sulphates of the cations sodium, calcium, potassium and/or magnesium are preferred.

For the generation of the pore structure the compartments are subsequently removed from the polysulfone matrix. The three dimensional structure with the predetermined grade of porosity remains, which can be then “loaded” with the active agent.

In the following three preferred systems for the generation of the pore structure are described shortly on the basis of the additive classes polymer, mineral and water.

System 1: Polymer

As polymeric additives are used e.g. special high (super) branched polyesters with thermally weak triazene groups in the major chain. The molecularly dispersed high (super) branched polymer is built into the polysulfone matrix. The subsequent thermal treatment of the system degradates the high (super) branched pore creator into volatile degradation products among the generation of a corresponding nanoporous polymer layer. Polysulfones distinguish themselves inter alia by their temperature stability and high dimension stability, whereby this strategy is applicable by all means. Moreover this thermal treatment can be coupled to the step of sterilization leading to an efficient method.

System 2: Mineral

As mineral additive e.g. the physiologically harmless compound calcium carbonate is used. The polysulfone matrix consist of double hydrophilic blockcopolymers. These double hydrophilic blockcopolymers comprise a hydrophilic block, which does not interact with the mineral additive, and a second polyelectrolyte block, which interacts strongly with the surfaces of the mineral additive. These blockcopolymers act during the crystallization of calcium carbonate growth modificatory. The resulting mineral compartments have an approximate oval, bar-bell or spherical shape. Due to the excellent resistence of the polysulfones towards aggressive chemicals as well as the hydrolysis stability the mineral additives can be removed completely in the acid bath. The desired nanoporous structure of the polysulfone matrix remains.

System 3: Water

As fluid additive in the case of the coating of the medical product with polar active agents water comes into consideration as easiest solution. During the use of the spraying method the polysulfone is present in an organic solvent such as chloroform. The polysulfone saturated chloroform solution is only conditionally capable of the further reception of the active agent. Thus the active agent is solved predominantly in the aqueous phase, which forms compartments due to the phase separation subsequent to the deposition on the surface of the medical product. Afterwards the water can be removed from this compartments e.g. by means of the freeze drying completely out of the system. The active agent loaded nanoporous structure remain. The active agent concentration of the pores can be increased in consecutive steps with active agent solved in water and preferably subsequent freeze drying. During the methods up to now also the active agent was solved together with the polysulfone in chloroform. The consecutive concentration of the active agent was also carried out from a chloroform solution. As the chloroform cannot be removed from the layer at no time with 100%, the chloroform increasingly concentrates in the completed end product, which leads to an unnecessary exposure of the patient. By using water as active agent carrier chloroform is used only one time for the deposition of the polysulfone matrix and the exposure is reduced to a minimum.

For the preparation of a spraying solution containing at least one polysulfone and at least one antiproliferative, antiinflammatory, antiphlogistic and/or antithrombotic active agent moreover preferred solvents are suitable, which evaporate easily, i.e. which are volatile such as chloroform, dichloromethane, tetrahydrofuran, acetone, methanol, ethanol, isopropanol, diethyl ether and ethyl acetate and which can be saturated moreover with water or be prepared with a particular water content. Thereby water contents from 1.6-15%, preferred 2.1-10%, more preferred 2.6-7.9% and especially preferred 3.3-6.8% are suitable. Further is preferred if organic solvent, water, polysulfone and active agent form a homogeneous solution.

Through the generation of copolymers the hydrophilic property resp. hydrophobic property of the polysulfone can be also varied. Polysulfone copolymers with 4,4′-bis(hydroxyphenyl)pentanoic acid (BPA) can be synthesized, for example, so that in doing so carboxylic side groups are introduced, which lower the hydrophobic property of the polysulfone matrix. Moreover there is now the possibility to derivatize introduced functional groups e.g. the carboxylic group (Macrom. Chem. Phys. 195 (1994), 1709; Coll. Polym. Sci. 279 (2001), 727).

Through the possibility of forming of at least two layers of the polymer, which is variable in its composition, as well as in the variation of the additives in addition a layer dependent differentiation in respect of the used active agents as well as in respect of the concentration can be conducted. This adaptation capability distinguishes the polysulfone matrix as an universally usable biostable coating material for preventing restenosis.

Further the use of thermoplastic polysulfones is preferred. Thermoplastic polysulfones can be deformed plastically (plastic) under the influence of heat (thermo). Normally thermoplastic polysulfones consist of linear or somewhat branches molecule chains. If heated they can be extended by stretching. If heated stronger they can be smelt completely and rebuilt. Especially it is preferred, if these thermoplastic polysulfones feature hydrophilic as well as hydrophobic properties. Such thermoplastic polysulfones with these ambivalent properties can be prepared according to the above described methods via polymer analogous reactions, blockcopolymerizations or polymerization of hydrophilic with hydrophobic monomers. The such obtained thermoplastic polymers resp. the therewith coated medical products distinguish themselves by multiple sterilisation ability, hot steam and hydrolysis resistence, high dimension stability, resistence towards aggressive chemicals as well as good thermal aging stability.

A preferred thermoplastic polysulfone is manufactured from bisphenol A and 4,4′-dichlorophenylsulfone via polycondensation reactions (see following formula (II)).

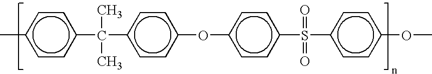

### Poly[oxy-1,4-phenylene-sulfonyl-1,4-phenylene-oxy-(4,4′-isopropylidenediphenylene)]

The polysulfones usable for the coating according to invention have the following general structure according to formula (I): 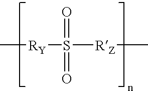
wherein
n represents the grade of polymerization, which is in the range of n=10 to n=10.000, preferred in the range of n=20 to n=3.000, more preferred in the range of n=40 to n=1.000, more preferred in the range of n=60 to n=500, more preferred in the range of n=80 to n=250 and especially preferred in the range of n=100 to n=200.

Further preferred is, if n is in such a range so that a weight average of the polymer results in 60.000-120.000 g/mol, preferred 70.000 to 99.000 g/mol, more preferred 80.000 to 97.000 g/mol, still more preferred 84.000 to 95.000 g/mol, and especially preferred 86.000 to 93.000 g/mol.

In addition it is preferred, if n is in such a range that the number average of the polymer results in a range of 20.000 to 70.000 g/mol, preferred 30.000 to 65.000 g/mol, more preferred 32.000 to 60.000 g/mol, still more preferred 35.000 to 59.000 g/mol, and especially preferred 45.000 to 58.000 g/mol.

Y and z are integer numbers in the range of 1 to 10, and R and R′ form independently from each other an alkylene group with 1 to 12 carbon atoms, an aromatic group with 6 to 20 carbon atoms, a heteroaromatic group with 2 to 10 carbon atoms, a cycloalkylene group with 3 to 15 carbon atoms, an alkylenearylene group with 6 to 20 carbon atoms, an arylenealkylene group with 6 to 20 carbon atoms, an alkyleneoxy group with 1 to 12 carbon atoms, an aryleneoxy group with 6 to 20 carbon atoms, a heteroaryleneoxy group with 6 to 20 carbon atoms, a cycloalkyleneoxy group with 3 to 15 carbon atoms, an alkylenearyleneoxy group with 6 to 20 carbon atoms or an arylenealkyleneoxy group with 6 to 20 carbon atoms. The aforementioned groups can bear further substituents especially those which are described more below under “substituted” polysulfones.

Examples for the groups R and R′ are —R¹—, —R²—, —R³—, —R⁴—, —R⁵—, —R⁶—, —R¹—R²—, —R³—R⁴—, —R⁵—R⁶—, —R¹—R²—R³—, —R⁴—R⁵—R⁶—, —R¹—R²—R³—R⁴—, —R¹—R²—R³—R⁴—R⁵— as well as —R¹—R²—R³—R⁴—R⁵—R⁶—;

wherein R¹, R², R³, R⁴, R⁵ and R⁶ represent independently from each other the following groups:

—CH₂—, —C₂H₄—, —CH(OH)—, —CH(SH)—, —CH(NH₂)—, —CH(OCH₃)—, —C(OCH₃)₂—, —CH(SCH₃)—, —C(SCH₃)₂—, —CH(NH(CH₃))—, —C(N(CH₃)₂)—, —CH(OC₂H₅)—, —C(OC₂H₅)₂—, —CHF—, —CHCl—, —CHBr—, —CF₂—, —CCl₂—, —CBr₂—, —CH(COOH)—, —CH(COOCH₃)—, —CH(COOC₂H₅)—, —CH(COCH₃)—, —CH(COC₂H₅)—, —CH(CH₃)—, —C(CH₃)₂—, —CH(C₂H₅)—, —C(C₂H₅)₂—, —CH(CONH₂)—, —CH(CONH(CH₃))—, —CH(CON(CH₃)₂)—, —C₃H₆—, —C₄H₈—, —C₅H₉—, —C₆H₁₀—, cyclo-C₃H₄—, cyclo-C₃H₄—, cyclo-C₄H₆—, cyclo-C₅H₈—, —OCH₂—, —OC₂H₄—, —OC₃H₆—, —OC₄H₈—, —OC₅H₉—, —OC₆H₁₀—, —CH₂O—, —C₂H₄O—, —C₃H₆O—, —C₄H₈O—, —C₅H₉O—, —C₆H₁₀O—, —NHCH₂—, —NHC₂H₄—, —NHC₃H₆—, —NHC₄H₈—, —NHC₅H₉—, —NHC₆H₁₀—, —CH₂N H—, —C₂H₄N H—, —C₃H₆N H—, —C₄H₈N H—, —C₅H₉NH—, —C₆H₁₀NH—, —SCH₂—, —SC₂H₄—, —SC₃H₆—, —SC₄H₈—, —SC₅H₉—, —SC₆H₁₀—, —CH₂S—, —C₂H₄S—, —C₃H₆S—, —C₄H₈S—, —C₅H₉S—, —C₆H₁₀S—, —C₆H₄—, —C₆H₃(CH₃)—, —C₆H₃(C₂H₅)—, —C₆H₃(OH)—, —C₆H₃(NH₂)—, —C₆H₃(Cl), —C₆H₃(F)—, —C₆H₃(Br)—, —C₆H₃(OCH₃)—, —C₆H₃(SCH₃)—, —C₆H₃(COCH₃)—, —C₆H₃(COC₂H₅)—, —C₆H₃(COOH)—, —C₆H₃(COOCH₃)—, —C₆H₃(COOC₂H₅)—, —C₆H₃(NH(CH₃))—, —C₆H₃(N(CH₃)₂)—, —C₆H₃(CONH₂)—, —C₆H₃(CONH(CH₃))—, —C₆H₃(CON(CH₃)₂)—, —OC₆H₄—, —OC₆H₃(CH₃)—, —OC₆H₃(C₂H₅)—, —OC₆H₃(OH)—, —OC₆H₃(N H₂)—, —OC₆H₃(Cl)—, —OC₆H₃(F)—, —OC₆H₃(Br)—, —OC₆H₃(OCH₃)—, —OC₆H₃(SCH₃)—, —OC₆H₃(COCH₃)—, —OC₆H₃(COC₂H₅)—, —OC₆H₃(COOH)—, —OC₆H₃(COOCH₃)—, —OC₆H₃(COOC₂H₅)—, —OC₆H₃(N H(CH₃))—, —OC₆H₃(N(CH₃)₂)—, —OC₆H₃(CONH₂)—, —OC₆H₃(CONH(CH₃))—, —OC₆H₃(CON(CH₃)₂)—, —C₆H₄O—, —C₆H₃(CH₃)O—, —C₆H₃(C₂H₅)O—, —C₆H₃(OH)O—, —C₆H₃(N H₂)O—, —C₆H₃(Cl)O—, —C₆H₃(F)O—, —C₆H₃(Br)O—, —C₆H₃(OCH₃)O—, —C₆H₃(SCH₃)O—, —C₆H₃(COCH₃)O—, —C₆H₃(COC₂H₅)O—, —C₆H₃(COOH)O—, —C₆H₃(COOCH₃)O—, —C₆H₃(COOC₂H₅)O—, —C₆H₃(N H(CH₃))O—, —C₆H₃(N(CH₃)₂)O—, —C₆H₃(CON H₂)O—, —C₆H₃(CONH(CH₃))O—, —C₆H₃(CON(CH₃)₂)O—, —SC₆H₄—, —SC₆H₃(CH₃)—, —SC₆H₃(C₂H₅)—, —SC₆H₃(OH)—, —SC₆H₃(N H₂)—, —SC₆H₃(Cl)—, —SC₆H₃(F)—, —SC₆H₃(Br)—, —SC₆H₃(OCH₃)—, —SC₆H₃(SCH₃)—, —SC₆H₃(COCH₃)—, —SC₆H₃(COC₂H₅)—, —SC₆H₃(COOH)—, —SC₆H₃(COOCH₃)—, —SC₆H₃(COOC₂H₅)—, —SC₆H₃(NH(CH₃), —SC₆H₃(N(CH₃)₂)—, —SC₆H₃(CONH₂)—, —SC₆H₃(CONH(CH₃))—, —SC₆H₃(CON(CH₃)₂)—, —C₆H₄S—, —C₆H₃(CH₃)S—, —C₆H₃(C₂H₅)S—, —C₆H₃(OH)S—, —C₆H₃(N H₂)S—, —C₆H₃(Cl)S—, —C₆H₃(F)S—, —C₆H₃(Br)S—, —C₆H₃(OCH₃)S—, —C₆H₃(SCH₃)S—, —C₆H₃(COCH₃)S—, —C₆H₃(COC₂H₅)S—, —C₆H₃(COOH)S—, —C₆H₃(COOCH₃)S—, —C₆H₃(COOC₂H₅)S—, —C₆H₃(N H(CH₃))S—, —C₆H₃(N(CH₃)₂)S—, —C₆H₃(CONH₂)S—, —C₆H₃(CONH(CH₃))S—, —C₆H₃(CON(CH₃)₂)S—, —NH—C₆H₄—, —NH—C₆H₃(CH₃)—, —NH—C₆H₃(C₂H₅)—, —NH—C₆H₃(OH)—, —NH—C₆H₃(NH₂)—, —NH—C₆H₃(Cl)—, —NH—C₆H₃(F)—, —NH—C₆H₃(Br)—, —NH—C₆H₃(OC H₃)—, —NH—C₆H₃(SCH₃)—, —NH—C₆H₃(COCH₃)—, —NH—C₆H₃(COC₂H₅)—, —NH—C₆H₃(COOH)—, —NH—C₆H₃(COOCH₃)—, —NH—C₆H₃(COOC₂H₅)—, —NH—C₆H₃(NH(CH₃))—, —NH—C₆H₃(N(CH₃)₂)—, —NH—C₆H₃(CON H₂)—, —NH—C₆H₃(CON H(CH₃))—, —NH—C₆H₃(CON(CH₃)₂)—, —C₆H₄—NH—, —C₆H₃(CH₃)—NH—, —C₆H₃(C₂H₅)—NH—, —C₆H₃(OH)—NH—, —C₆H₃(N H₂)—NH—, —C₆H₃(CH₃)—NH—, —C₆H₃(F)—NH—, —C₆H₃(Br)—NH—, —C₆H₃(OCH₃)—NH—, —C₆H₃(SCH₃)—NH—, —C₆H₃(COCH₃)—NH—, —C₆H₃(COC₂H₅)—NH—, —C₆H₃(COOH)—NH—, —C₆H₃(COOCH₃)—NH—, —C₆H₃(COOC₂H₅)—NH—, —C₆H₃(NH(CH₃))—NH—, —C₆H₃(N(CH₃)₂)—NH—, —C₆H₃(CON H₂)—NH—, —C₆H₃(CON H(CH₃))—NH—, —C₆H₃(CON(CH₃)₂)—NH—.

Especially preferred are polysulfones as well as their mixtures wherein the groups —R¹—, —R²—, —R³—, —R¹—R²—, —R¹—R²—R³— represent independently from each other the following groups: —C₆H₄O—, —C(CH₃)₂—, —C₆H₄—, —C₆H₄SO₂—, —SO₂C₆H₄—, —OC₆H₄—, and —C₆H₄O—C(CH₃)₂—C₆H₄—.

R and R′ can represent further preferred independently from each other a group, which is bound to the sulfone group in the formulas (II) to (XV).

According to invention the polysulfone and the polysulfones respectively for the biostable layer or the biostable layers are selected from the group comprising: polyethersulfone, substituted polyethersulfone, polyphenylsulfone, substituted polyphenylsulfone, polysulfone block copolymers, perfluorinated polysulfone block copolymers, semifluorinated polysulfone block copolymers, substituted polysulfone block copolymers and/or mixtures of the aforementioned polymers.

The term “substituted” polysufones is representative for polysulfones which bear functional groups. Especially the methylene units can feature one or two substituents and the phenylene units one, two, three, or four substituents. Examples for these substituents (also referred to as: X, X′, X″, X′″) are:
—OH, —OCH₃, —OC₂H₅, —SH, —SCH₃, —SC₂H₅, —NO₂, —F, —Cl, —Br, —I, —N₃, —CN, —OCN, —NCO, —SCN, —NCS, —CHO, —COCH₃, —COC₂H₅, —COOH, —COCN, —COOCH₃, —COOC₂H₅, —CONH₂, —CONHCH₃, —CONHC₂H₅, —CON(CH₃)₂, —CON(C₂H₅)₂, —NH₂, —NHCH₃, —NHC₂H₅, —N(CH₃)₂, —N(C₂H₅)₂, —SOCH₃, —SOC₂H₅, —SO₂CH₃, —SO₂C₂H₅, —SO₃H, —SO₃CH₃, —SO₃C₂H₅, —OCF₃, —O—COOCH₃, —O—COOC₂H₅, —NH—CO—NH₂, —NH—CS—NH₂, —NH—C(═NH)—NH₂, —O—CO—NH₂, —NH—CO—OCH₃, —NH—CO—OC₂H₅, —CH₂F—CHF₂, —CF₃, —CH₂Cl —CHCl₂, —CCl₃, —CH₂Br —CHBr₂, —CBr₃, —CH₂₁—CH₁₂, —Cl₃, —CH₃, —C₂H₅, —C₃H₇, —CH(CH₃)₂, —C₄H₉, —CH₂—CH(CH₃)₂, —CH₂—COOH, —CH(CH₃)—C₂H₅, —C(CH₃)₃, —H.

Further preferred substituents or functional groups are —CH₂—X and —C₂H₄—X.

The following general structural formula represent preferred repeating units for polysulfones. Preferred the polymers consist only of these repeating units. It is also possible, that in one polymer besides the shown repeating units other repeating units or blocks are present. Preferred are:

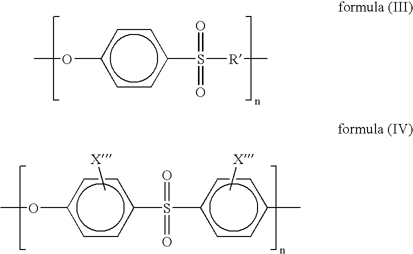

X, X′, n and R′ have independently from each other the above mentioned meaning.

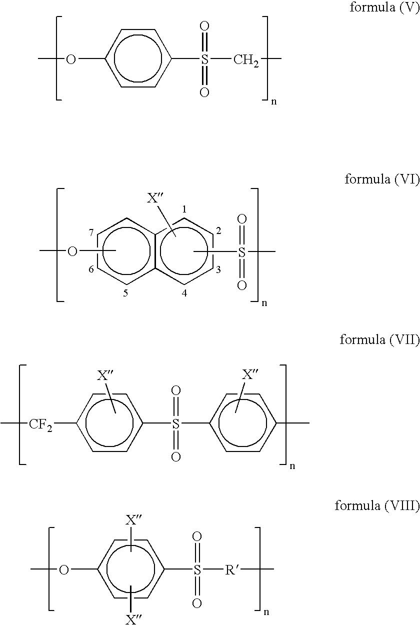

X, X′, n and R′ have independently from each other the above mentioned meaning.

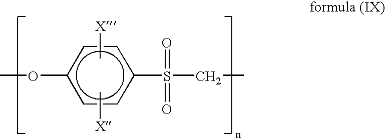

Further are preferred polysulfones of the following general formula (X):

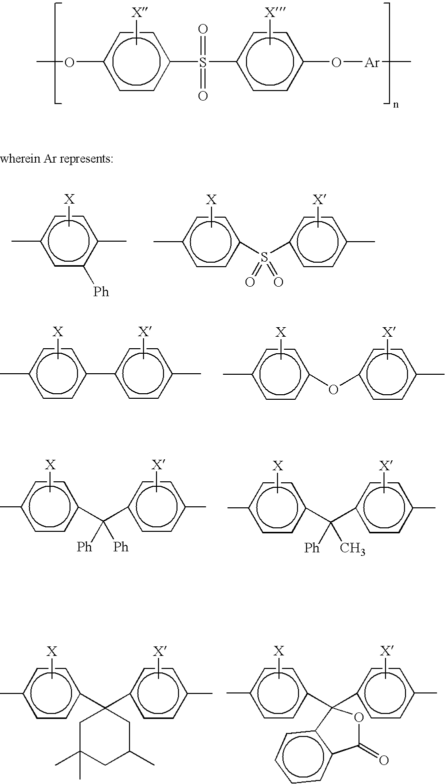

X, X′ and n have independently from each other the above mentioned meaning.

The following repeating units are further preferred:

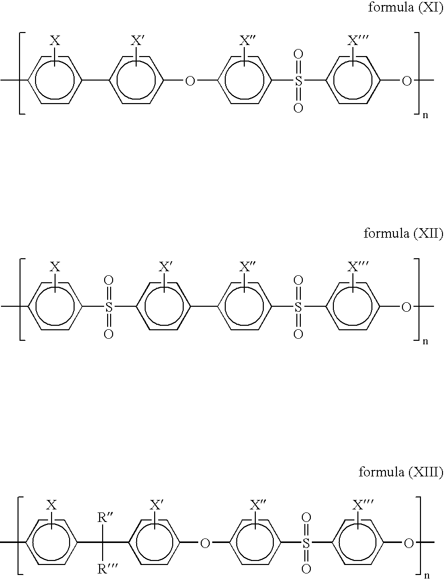

X, X′, X″, X′″ and n have independently from each other the above mentioned meaning. R″ and R′″ can represent independently from each other a substituent as it is defined for X or X′ or can represent independently from each other a group —R¹—H or —R²—H.

A further preferred repeating unit features a cyclic substituent between two aromatic rings such as formula (XIV) or (XV):

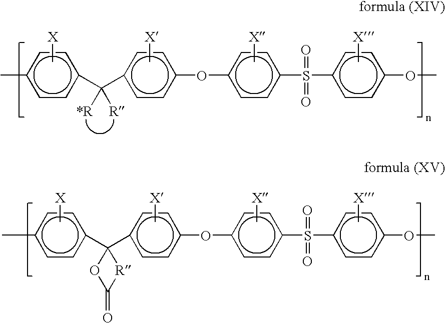

R″ represents preferred —CH₂—, —OCH₂—, —CH₂O—, —O—, —C₂H₄—, —C₃H₆—, —CH(OH)—. The group —*R—R″— represents preferred a cyclic ester, amide, carbonate, urea or urethane such as: —O—CO—O—, —O—CO—O—CH₂—, —O—CO—O—C₂H₄—, —CH₂—O—CO—O—CH₂—, —C₂H₄—, —C₃H₆—, —C₄H₈—, —C₅H₁₀—, —C₆H₁₂—, —O—CO—NH—, —NH—CO—NH—, —O—CO—NH—CH₂—, —O—CO—NH—C₂H₄—, —NH—CO—NH—CH₂—, —NH—CO—NH—C₂H₄—, —NH—CO—O—CH₂—, —NH—CO—O—C₂H₄—, —CH₂—O—CO—NH—CH₂—, —C₂H₄—SO₂—, —C₃H₆—SO₂—, —C₄H₈—SO₂—, —C₂H₄—SO₂—CH₂—, —C₂H₄—SO₂—C₂H₄—, —C₂H₄—O—, —C₃H₆—O—, —C₄H₈—O—, —C₂H₄—O—CH₂—, —C₂H₄—O—C₂H₄—, —C₂H₄—CO—, —C₃H₆—CO—, —C₄H₈—CO—, —C₂H₄—CO—CH₂—, —C₂H₄—CO—C₂H₄—, —O—CO—CH₂—, —O—CO—C₂H₄—, —O—CO—C₂H₂—, —CH₂—O—CO—CH₂—, or cyclic esters, which contain an aromatic ring.

In the following polymer analogous reactions will be described, which are known to the one skilled in the art and serve for the modification of the polysulfones.

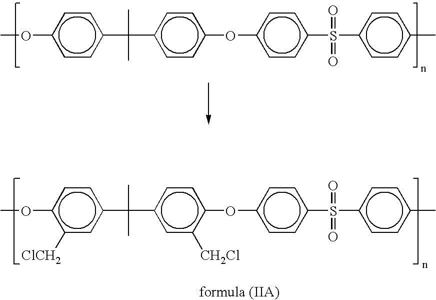

Chloromethylene groups as groups X and X′ can be introduced by using formaldehyde, ClSMe₃ and a catalyst such as SnCl₄, which then can be further substituted. Via these reactions e.g. hydroxyl groups, amino groups, carboxylate groups, ether or alkyl groups can be introduced by a nucleophilic substitution, which are bound by a methylene group to the aromatic residue. A reaction with alcoholates such as a phenolate, benzylate, methanolate, ethanolate, propanolate or isopropanolate leads to a polymer in which at over 75% of the chloromethylene groups a substitution took place. The following polysulfone with lipophilic side groups results: 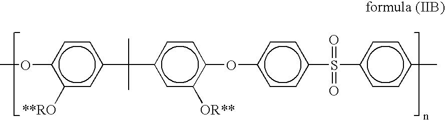
wherein
R** e.g. represents an alkyl group or aryl group.

The groups X″ and X′″ can be introduced, as far as not yet present in the monomers, at the polymer by the following reaction:

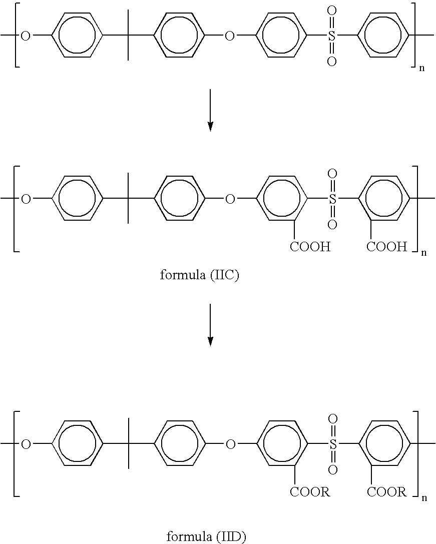

Besides an ester group other diverse substituents can be introduced in that first a single or double deprotonation is carried out with a strong base e.g. n-BuLi or tert-BuLi and subsequently an electrophile is added. In the above case of example carbon dioxide was added for the introduction of the ester group and the obtained carbonic acid group was esterified in another step.

A combination according to invention from a polysulfone with lipophilic groups and a polysulfone with lipophobic groups is achieved exemplary by using of polysulfone according to formula (IIB) together with polysulfone according to formula (IIC). The amount ratios of both polysulfones to each other can range from 98%:2% to 2%:98%. Preferred areas are 10% to 90%, 15% to 85%, 22% to 78% and 27% to 73%, 36% to 64%, 43% to 57% and 50% to 50%. These percentage indications are to be applied for any combinations of hydrophilic and hydrophobic polysulfones and are not limited to the above-mentioned mixture.

An example for a polysulfone with hydrophilic and hydrophobic groups in one molecule can be obtained for example in that the polysulfone according to formula (IIC) is only esterified incompletely and thus hydrophilic carboxylate groups and hydrophobic ester groups are present in one molecule. The mole ratio (number) of carboxylate groups to ester groups can be 5%:95% to 95%:5%. These percentage indications are to be applied for any combinations of hydrophilic and hydrophobic groups and are not limited to the above-mentioned ones.

It is supposed, that by this combination according to invention of hydrophilic groups resp. polymers with hydrophobic groups resp. polymers amorphous polymer layers are built on the medical product. It is very important, that the polymer layers of polysulfone may not be crystalline or predominantly crystalline, as crystallinity leads to rigid layers, which break down and separate themselves. Flexible polysulfone coatings, which serve as a barrier layer, can be achieved only with amorphous or predominantly amorphous polysulfone layers.

Of course it is also possible to use already correspondingly substituted monomers so as to obtain the desired substitution pattern after the effected polymerization. The corresponding polymers result then in the known manner according to the following reaction scheme: 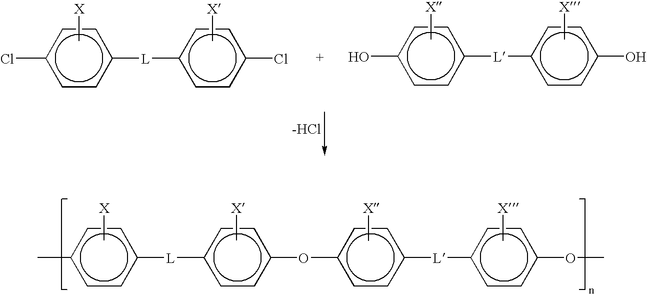
wherein
L and L′ independently from each other exemplary represent the following groups: —SO₂—, —C(CH₃)₂—, —C(Ph)₂— or —O—. L and L′ can thus have the meanings of the corresponding groups in the formulas (I) to (XV). Such nucleophilic substitution reactions are known to the one skilled in the art, which are illustrated exemplary by the above scheme.

As already mentioned it is especially preferred, if the polymers feature hydrophilic and hydrophobic properties, on the one hand within one polymer and on the other hand by using at least one hydrophilic polymer in combination with at least one hydrophobic polymer. Thus it is preferred, if for example in the case of X and X′ hydrophilic substituents and in the case of X″ and X′″ hydrophobic substituents are concerned, or vice versa.

As hydrophilic substituents come into question: —OH, —CHO, —COOH, —COO⁻, —CONH₂, —NH₂, —N⁺(CH₃)₄, —NHCH₃, —SO₃H, —SO₃⁻, —NH—CO—NH₂, —NH—CS—NH₂, —NH—C(═NH)—NH₂, —O—CO—NH₂ and especially protonated amino groups.

As hydrophobic substituents come into question: —H, —OCH₃, —OC₂H₅, —SCH₃, —SC₂H₅, —NO₂, —F, —Cl, —Br, —I, —N₃, —CN, —OCN, —NCO, —SCN, —NCS, —COCH₃, —COC₂H₅, —COCN, —COOCH₃, —COOC₂H₅, —CONHC₂H₅, —CON(CH₃)₂, —CON(C₂H₅)₂, —NHC₂H₅, —N(CH₃)₂, —N(C₂H₅)₂, —SOCH₃, —SOC₂H₅, —SO₂CH₃, —SO₂C₂H₅, —SO₃CH₃, —SO₃C₂H₅, —OCF₃, —O—COOCH₃, —O—COOC₂H₅, —NH—CO—OCH₃, —NH—CO—OC₂H₅, —CH₂F —CHF₂, —CF₃, —CH₂Cl —CHCl₂, —CCl₃, —CH₂Br —CHBr₂, —CBr₃, —CH₂₁—CH₁₂, —Cl₃, —CH₃, —C₂H₅, —C₃H₇, —CH(CH₃)₂, —C₄H₉, —CH₂—CH(CH₃)₂, —CH₂—COOH, —CH(CH₃)—C₂H₅, —C(CH₃)₃.

Moreover cyclic polysulfones are preferred, which feature for example a structure as shown in formula (XVI):

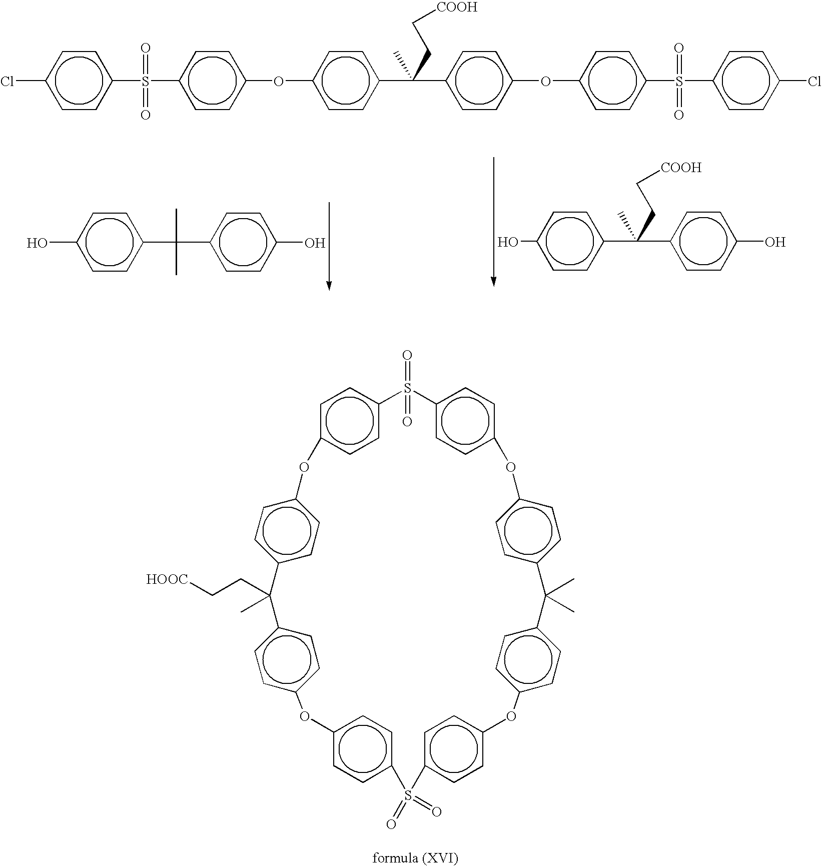

The carboxyethylene group is not essential for the above exemplary reaction. Instead of the carboxyethylene and the methyl substituents any other substituents or also hydrogen can be present.

Polysulfones are characterized by their high resistence against aggressive chemicals, they are stable to hydrolysis and heat and possess very good mechanical and tribological (no surface wear) properties. As further special properties as material for the use in the living organism the high dimension stability and the multiple sterilization can be accentuated. Polysulfones are used already for a long time as medical polymers. The main use concentrates on hollow fibres e.g. in blood dialyzers where the polysulfone fibres from the Fresenius company are leading on the global market due to their good hemocompatibility and membrane forming properties. The problem of dialysis consists primarily in the necessity that during hemodialysis an anticoagulant normally heparin has to be administered which side effects prevalence after a couple of years. About 75 litres of blood—this is equivalent to about the 15-times present blood amount of the patient—flow during a five hour treatment through the dialyzer. Therewith it is clear, that a very high requirement of hemocompatibility is set to the membrane.

Another large area is the use of polysulfone capillaries in ophthalmology and in form of flat membranes in various medical technologic auxiliary means.

It is preferred when the polysulfone used for the biostable layer is added at least one hydrophilic polymer. Thereby the ratio of polysulfone to hydrophilic polymer can be 50% by weight to 50% by weight up to 99.999% by weight to 0.001% by weight in the respective polysulfone layer.

As hydrophilic polymers are suitable polyvinylpyrrolidone, glycerine, polyethylene glycol, polypropylene glycol, polyvinyl alcohol, polyhydroxyethyl methacrylates, polyacrylamide, polyvalerolactones, poly-ε-decalactones, polylactic acid, polyglycolic acid, polylactides, polyglycolides, copolymers of the polylactides and polyglycolides, poly-ε-caprolactone, polyhydroxybutanoic acid, polyhydroxybutyrates, polyhydroxyvalerates, polyhydroxybutyrate-co-valerates, poly(1,4-dioxane-2,3-diones), poly(1,3-dioxane-2-ones), poly-p-dioxanones, polyanhydrides such as polymaleic anhydrides, fibrin, polycyanoacrylates, polycaprolactonedimethylacrylates, poly-b-maleic acid, polycaprolactone butylacrylates, multiblock polymers such as from oligocaprolactonedioles and oligodioxanonedioles, polyether ester multiblock polymers such as PEG and polybutylene terephthalate, polypivotolactones, polyglycolic acid trimethyl-carbonates, polycaprolactone-glycolides, poly-g-ethylglutamate, poly(DTH-iminocarbonate), poly(DTE-co-DT-carbonate), poly(bisphenol-A-iminocarbonate), polyorthoesters, polyglycolic acid trimethyl-carbonates, polytrimethylcarbonates, polyiminocarbonates, poly(N-vinyl)-pyrrolidone, polyvinylalcohols, polyesteramides, glycolated polyesters, polyphosphoesters, polyphosphazenes, poly[p-carboxyphenoxy)propane], polyhydroxypentanoic acid, polyanhydrides, polyethyleneoxide-propyleneoxide, soft polyurethanes, polyurethanes with amino acid residues in the backbone, polyether esters such as polyethyleneoxide, polyalkeneoxalates, polyorthoesters as well as copolymers thereof, lipids, carrageenans, fibrinogen, starch, collagen, protein based polymers, polyamino acids, synthetic polyamino acids, zein, modified zein, polyhydroxyalkanoates, pectic acid, actinic acid, modified and non modified fibrin and casein, carboxymethyl sulphate, albumin, hyaluronic acid, chitosan and its derivatives, chondroitine sulphate, dextran, b-cyclodextrins, copolymers with PEG and polypropylene glycol, gum arabicum, guar, gelatine, collagen, collagen-N-hydroxysuccinimide, lipids, phospholipids, modifications and copolymers and/or mixtures of the afore mentioned substances, polyvinylpyrrolidone polyethylene glycol and glycerine are used preferably.

For increasing the viscosity in the production of the polysulfone solution e.g. polyvinylpyrrolidone (PVP) is added which is soluble in the precipitation agent during the manufacture of the hollow fibres and is thereby removed. The completed porous hollow fibre still contains an amount of 1-2% PVP in average. The addition of polyvinylpyrrolidone is not only beneficial for the viscosity during the production, i.e. increases viscosity, but also a factor co-determining the pore size of the polysulfone and thereby decisive for the permeability properties of the end product, for being dependent from the pore size and the particle size. Thus the pore size and thereby the permeability of the produced polysulfone can be regulated via the amount and the molecular weight of the admixed polyvinylpyrrolidone.

The biocompatible and good mechanical properties of polysulfone and the possibility for controlling the pore size by the addition of polyvinylpyrrolidone and/or another hydrophilic polymer and/or water (ethyl acetate) turns this polymer into the ideal substrate for all pharmaceutics, which can be used for the targeted local application such as in cardiology for the prevention of restenosis. Simultaneously the occluded nitrogen takes care of the shelf life of the active agent. The preferred amount of the added polymer is in the range of 0.5 to 50% by weight, further preferred are 1 to 20% by weight, especially preferred are 2 to 10% by weight. The added amount complies substantially with the desired elution velocity of the used active agent.

The medical products according to invention possess a surface which can be made of any material. This surface is preferably not hemocompatible. Further this surface is preferably not coated, especially not with polymers and/or organic macromolecules.

The biostable polysulfone layer can be bound adhesively or covalently as well as partially adhesively and partially covalently to this surface. Preferred is the covalent bonding. The polysulfone layer covers the surface of the medical product at least partially, preferably completely. If the medical product is a stent, at least the surface exposed to the blood is covered with polysulfone.

Preferably at least one layer containing at least one antiproliferative, antiinflammatory, antiphlogistic and/or antithrombotic active agent can be deposited and/or incorporated on this first biostable polysulfone layer and/or into this first polysulfone layer. The at least one layer containing at least one antiproliferative, antiinflammatory, antiphlogistic and/or antithrombotic active agent can completely consist of one or more active agents or can be another biostable polysulfone layer, wherein the active agent or the active agents are located, or can be a hemocompatible layer, wherein the active agent or the active agents are located. Whilst hydrophobic active agents can be deposited in and/or on and/or under a biostable layer, hydrophilic active agents are preferably deposited on and/or under a biostable layer.

Thus the medical products according to invention can feature surfaces, which are coated with one, two, three or more layers, one, two or three layers and especially two layers are preferred.

The antiproliferative, antiinflammatory, antiphlogistic and/or antithrombotic active agent(s) can be bound on the respective layer adhesively or covalently or in part adhesively and in part covalently, the adhesive bonding is preferred.

If the surface coating features more biostable polysulfone layers and/or hemocompatible layers and/or active agent layers each of these layers can consist of different polysulfones with different hydrophilic polymers and different amounts of hydrophilic polymers as well as different hemocompatible compounds or different active agents.

Further preferred is, when the medical product features a surface, which comprises a hemocompatible layer, which is deposited and/or incorporated on or in the lowest first biostable polysulfone layer. This hemocompatible layer can also form a second or third layer, which lies directly or indirectly on the lowest biostable layer and/or on or under an active agent layer or a second biostable polysulfone layer. Moreover preferred is, when the hemocompatible layer forms the lowest layer covered by an active agent layer covered in turn by a biostable polysulfone layer or when a biostable polysulfone layer with an active agent or an active agent combination is deposited on the lowest hemocompatible layer.

This hemocompatible layer consists preferably of completely desulphated and N-reacetylated heparin, desulphated and N-reacetylated heparin, N-carboxymethylated, partially N-acetylated chitosan and/or mixtures of these substances. The hemocompatible layer can comprise besides the aforementioned substances other hemocompatible organic substances, but consists preferably only of the aforementioned substances.

Preferred is in case of the medical products according to invention, when only one hemocompatible layer is present. Furthermore, preferred is, if this hemocompatible layer forms the external or the lowest layer.

Further is preferred, that a layer completely covers the subjacent surface or the subjacent layer, while a partial cover is also possible.

Further is especially preferred, if the medical product according to invention is a stent. This stent can be formed of any material and material compositions. Preferred are metals and polymers such as medical stainless steel, titanium, chromium, vanadium, tungsten, molybdenum, gold and nitinol. Preferably the stent is uncoated and/or not or only conditionally hemocompatible. Especially the stent does not bear a coating of organic material. Medical wires can be excluded as medical products.

These stents according to invention are preferably provided with at least one biocompatible biostable polysulfone layer covering the stent completely or incompletely with or without a defined ratio of a hydrophilic polymer and with at least one antiproliferative, antiinflammatory, antiphlogistic and/or antithrombotic active agent. Thereby the active agent can be present in the matrix and/or cover the matrix as second layer. In this context the second layer is referred to as the layer deposited on the first layer, etc.

Another preferred embodiment of the stents according to invention features a coating, which consists of at least two polysulfone layers. According to this dual layer embodiment the first layer consists of a layer, which is covered substantially completely by another biostable layer of the same or different pore size. One or both layers contain at least one antiproliferative, antiinflammatory, antiphlogistic and/or antithrombotic active agent. Similarly used are active agent combinations, which mutually support and/or complement each other in their properties.

Based on this dual layer embodiment there is the possibility to incorporate different active agents separated from each other in the respective layer suitable for the respective active agent, so that a hydrophobic active agent is located in the one more hydrophilic layer and shows another elution kinetics as another hydrophobic active agent, which is located in the more hydrophobic polymer layer or vice versa, for example. This offers a broad field of possibilities to place the availability of the active agents in a distinct reasonable sequence as well as to control the elution time and concentration.

Another preferred embodiment of the stents according to invention features a coating, which consists of at least three layers. According to this triple layer embodiment the first layer consists of a layer, which is covered substantially completely or incompletely by another second layer of pure active agent or active agent combinations, which in turn is covered by a third biostable polysulfone layer of same or different pore size. The polysulfone layers contain either no active agent or one or both represent matrices for at least one antiproliferative, antiinflammatory, antiphlogistic and/or antithrombotic active agent. Also used are active agent combinations, which mutually support and/or complement each other in their properties.

This embodiment is especially suitable for the use of hydrophilic active agents or active agent combinations in the form of a pure active agent layer. The adjacent biostable polymer layer with a defined content of hydrophilic polymer serves for controlled elution the active agent. Active agent combinations with at least one hydrophilic active agent result in different elution kinetics.

Also the hydrophilic polymer which can de admixed to the also subjacent polysulfone can be used as topcoat.

The biocompatible coating of a stent provides for the necessary hemocompatibility and the active agent (or active agent combination), which is equally spread over the total surface of the stent, effects, that the ongrowth of the stent surface with cells, especially the smooth muscle cells and endothelic cells, takes place in a controlled manner. Thus, rapid ongrowth and overgrowth with cells on the stent surface does not take place, which could lead to restenosis, however the ongrowth with cells on the stent surface is not completely prevented by a high concentration of a medicament, which involves the danger of a thrombosis.

Thus, the use of polysulfone guarantees, that the active agent or the active agent combination incorporated adhesively on the subjacent layer and/or adhesively in the layer is released continuously and in small dosages, so that the ongrowth with cells on the stent surface is not prevented, but an overgrowth. This combination of both effects gives the stent according to invention the ability to grow rapidly into the vessel wall and reduces the risk of a restenosis as well as the risk of a thrombosis. The release of the active agent(s) takes place over a period of time from 1 to 24 months, preferred over 1 to 12 months after implantation, especially preferred 1 to 3 months after implantation.

The release of the active agent can be adapted through the regulation of the pore size with the addition of polyvinylpyrrolidone or another similar hydrophilic polymer, so that the individual characteristics of the active agent, the elution rate as well as its pharmacological kinetics and in the case of more than one active agent also the elution sequence can fulfil the required demand.

As active agents are used antiproliferative substances, antiphlogistical as well as antithrombotic agents. Preferably cytostatics, macrolide antibiotics and/or statins are used as antiproliferative active agents. Applyable antiproliferative active agents are sirolimus (rapamycin), everolimus, pimecrolimus, somatostatin, tacrolimus, roxithromycin, dunaimycin, ascomycin, bafilomycin, erythromycin, midecamycin, josamycin, concanamycin, clarithromycin, troleandomycin, folimycin, cerivastatin, simvastatin, lovastatin, fluvastatin, rosuvastatin, atorvastatin, pravastatin, pitavastatin, vinblastine, vincristine, vindesine, vinorelbine, etoposide, teniposide, nimustine, carmustine, lomustine, cyclophosphamide, 4-hydroxycyclophosphamide, estramustine, melphalan, betulinic acid, camptothecin, lapachol, β-lapachone, podophyllotoxin, betulin, trofosfamide, podophyllic acid 2-ethylhydrazide, ifosfamide, chlorambucil, bendamustine, dacarbazine, busulfan, procarbazine, treosulfan, temozolomide, thiotepa, daunorubicin, doxorubicin, aclarubicin, epirubicin, mitoxantrone, idarubicin, bleomycin, mitomycin, dactinomycin, methotrexate, fludarabine, fludarabine-5′-dihydrogenphosphate, mofebutazone, acemetacin, diclofenac, lonazolac, dapsone, o-carbamoylphenoxyacetic acid, lidocaine, ketoprofen, mefenamic acid, piroxicam, meloxicam, chloroquine phosphate, penicillamine, hydroxychloroquine, auranofin, sodium aurothiomalate, oxaceprol, celecoxib, β-sitosterin, ademetionine, myrtecaine, polidocanol, nonivamide, levomenthol, benzocaine, aescin, cladribine, mercaptopurine, thioguanine, cytarabine, fluorouracil, gemcitabine, capecitabine, docetaxel, carboplatin, cisplatin, oxaliplatin, amsacrine, irinotecan, topotecan, hydroxycarbamide, miltefosine, pentostatin, aldesleukin, tretinoin, asparaginase, pegaspargase, anastrozole, exemestane, letrozole, formestane, aminoglutethimide, adriamycin, azithromycin, spiramycin, cepharantin, smc proliferation inhibitor-2w, epothilone A and B, mitoxantrone, azathioprine, mycophenolatmofetil, c-myc-antisense, b-myc-antisense, selectin (cytokine antagonist), CETP inhibitor, cadherines, cytokinin inhibitors, COX-2 inhibitor, NFkB, angiopeptin, ciprofloxacin, camptothecin, fluroblastin, monoclonal antibodies, which inhibit the muscle cell proliferation, bFGF antagonists, probucol, prostaglandins, folic acid and derivatives, vitamins of the B-row, vitamin D derivatives such as calcipotriol and tacalcitol, thymosine α-1, fumaric acid and its derivatives such as dimethylfumarate, IL-1β inhibitor, colchicine, NO donors such as pentaerythritol tetranitrate and syndnoeimines, S-nitrosoderivatives, tamoxifen, staurosporine, β-estradiol, α-estradiol, estrone, estriol, ethinylestradiol, fosfestrol, medroxyprogesterone, estradiol cypionates, estradiol benzoates, tranilast, kamebakaurin and other terpenoids, which are applied in the therapy of cancer, verapamil, tyrosine kinase inhibitors (tyrphostines), cyclosporine A, paclitaxel and derivatives thereof (6-α-hydroxy-paclitaxel, baccatin, taxotere and other), synthetically produced as well as from native sources obtained macrocyclic oligomers of carbon suboxide (MCS) and derivatives thereof, molgramostim (rhuGM-CSF), peginterferon α-2b, lenograstim (r-HuG-CSF), filgrastim, macrogol, dacarbazine, basiliximab, daclizumab, ellipticine, D-24851 (Calbiochem), colcemid, cytochalasin A-E, indanocine, nocodazole, S 100 protein, PI-88, melanocyte stimulating hormone (α-MSH), bacitracin, vitronectin receptor antagonists, azelastine, guanidyl cyclase stimulator, tissue inhibitor of metal proteinase-1 and -2, free nucleic acids, nucleic acids incorporated into virus transmitters, DNA and RNA fragments, plasminogen activator inhibitor-1, plasminogen activator inhibitor-2, antisense oligonucleotides, VEGF inhibitors, called IGF-1. From the group of antibiotics furthermore cefadroxil, cefazolin, cefaclor, cefotaxim, tobramycin, gentamycin are used. Positive influence on the postoperative phase have also the penicillins such as dicloxacillin, oxacillin, sulfonamides, metronidazol, antithrombotics such as argatroban, aspirin, abciximab, synthetic antithrombin, bivalirudin, coumadin, enoxaparin, hemoparin® (desulphated and N-reacetylated heparin), tissue plasminogen activator, GpIIb/IIIa platelet membrane receptor, factor Xa inhibitor, activated protein C, antibodies, heparin, hirudin, r-hirudin, PPACK, protamin, prourokinase, streptokinase, warfarin, urokinase, vasodilators such as dipyramidole, triazolopyrimidine (trapidil®), nitroprussides, PDGF antagonists such as triazolopyrimidine and seramin, ACE inhibitors such as captopril, cilazapril, lisinopril, enalapril, losartan, thiolprotease inhibitors, caspase inhibitors, apoptosis inhibitors, apoptosis regulators such as p65 NF-kB or Bcl-xL antisense oligonucleotides and prostacyclin, vapiprost, α, β and γ interferon, histamine antagonists, serotonin blockers, halofuginone, nifedipine, tocopherol, tranilast, molsidomine, tea polyphenols, epicatechin gallate, epigallocatechin gallate, Boswellic acids and derivatives thereof, leflunomide, anakinra, etanercept, sulfasalazine, etoposide, dicloxacillin, tetracycline, triamcinolone, mutamycin, procainamid, retinoic acid, quinidine, disopyramide, flecainide, propafenone, sotalol, amidorone. Further active agents are steroids (hydrocortisone, betamethasone, dexamethasone), non-steroidal substances (NSAIDS) such as fenoprofen, ibuprofen, indomethacin, naproxen, phenylbutazone and others. Antiviral agents such as acyclovir, ganciclovir and zidovudine are also applyable. Different antimycotics are used in this area. Examples are clotrimazole, flucytosine, griseofulvin, ketoconazole, miconazole, nystatin, terbinafine. Antiprozoal agents such as chloroquine, mefloquine, quinine are effective active agents in equal measure, moreover natural terpenoids such as hippocaesculin, barringtogenol-C21-angelate, 14-dehydroagrostistachin, agroskerin, agrostistachin, 17-hydroxyagrostistachin, ovatodiolids, 4,7-oxycycloanisomelic acid, baccharinoids B1, B2, B3, tubeimoside, bruceanol A, B and C, bruceantinoside C, yadanziosides N and P, isodeoxyelephantopin, tomenphantopin A and B, coronarin A, B, C and D, ursolic acid, hyptatic acid A, zeorin, iso-iridogermanal, maytenfoliol, effusantin A, excisanin A and B, longikaurin B, sculponeatin C, kamebaunin, leukamenin A and B, 13,18-dehydro-6-α-senecioyloxychaparrin, 1,11-dimethoxycanthin-6-one, 1-hydroxy-11-methoxycanthin-6-one, scopoletin, taxamairin A and B, regenilol, triptolide, moreover cymarin, apocymarin, aristolochic acid, anopterin, hydroxyanopterin, anemonin, protoanemonin, berberine, cheliburin chloride, cictoxin, sinococuline, bombrestatin A and B, cudraisoflavone A, curcumin, dihydronitidine, nitidine chloride, 12-β-hydroxypregnadiene-4,16-diene-3,20-dione, bilobol, ginkgol, ginkgolic acid, helenalin, indicine, indicine-N-oxide, lasiocarpine, inotodiol, glycoside 1a, podophyllotoxin, justicidin A and B, larreatin, malloterin, mallotochromanol, isobutyrylmallotochromanol, maquiroside A, marchantin A, maytansine, lycoridicin, margetine, pancratistatin, liriodenine, oxoushinsunine, aristolactam-AII, bisparthenolidine, periplocoside A, ghalakinoside, ursolic acid, deoxypsorospermin, psychorubin, ricin A, sanguinarine, manwu wheat acid, methylsorbifolin, sphatheliachromen, stizophyllin, mansonine, strebloside, akagerine, dihydrousambarensine, hydroxyusambarine, strychnopentamine, strychnophylline, usambarine, usambarensine, berberine, liriodenine, oxoushinsunine, daphnoretin, lariciresinol, methoxylariciresinol, syringaresinol, umbelliferon, afromoson, acetylvismione B, desacetylvismione A, vismione A and B, further natural terpenoids such as hippocaesculin, 14-dehydroagrostistachin, c-type natriuretic peptide (CNP) agroskerin, agrostistachin, 17-hydroxyagrostistachin, ovatodiolids, 4,7-oxycycloanisomelic acid, yadanziosides N and P, isodeoxyelephantopin, tomenphantopin A and B, coronarin A, B, C and D, ursolic acid, hyptatic acid A, zeorin, iso-iridogermanal, maytenfoliol, effusantin A, excisanin A and B, longikaurin B, sculponeatin.

The active agents are used separately or combined in the same or a different concentration. Especially preferred are active agents which feature also immunosuppressive properties besides their antiproliferative effect. Suchlike active agents are erythromycin, midecamycin, tacrolimus, sirolimus, paclitaxel and its derivatives and josamycin as well as triazolopyrimidine (trapidil®), D-24851, α- and β-estradiol, macrocyclic carbon suboxide (MCS) and its derivatives, PI-88, sodium salt of 2-methylthiazolidine-1,4-dicarboxylic acid and derivatives, and sirolimus. Furthermore preferred is a combination of several antiproliferatively acting substances or of antiproliferative active agents with immunosuppressive active agents.

Especially preferred are the active agents selected from the group comprising paclitaxel and its derivatives, β-estradiol, simvastatin, PI-88 (sulphated oligosaccharide; Progen Ind.), macrocyclic carbon suboxides (MCS) and their derivatives, triazolopyrimidine (trapidil®), N-(pyridine-4-yl)-[1-4-(4-chlorobenzyl)-indol-3-yl]-glyoxylamide (D-24851), and tacrolimus.

The active agent is preferably contained in a pharmaceutical active concentration from 0.001 to 20 mg per cm2 stent surface, further preferred 0.005 to 15 and especially preferred 0.01 to 10 mg per cm2 stent surface. Additional active agents can be contained in a similar concentration in the same or in other layers. Also preferred is an embodiment, which contains two different active agents in the same layer or in different layers. Further preferred is an embodiment, which features a pure active agent layer as supreme layer.

The amounts of polymer deposited on each medical product and especially on each stent are per layer preferred in the range between 0.01 mg/cm2 to 3 mg/cm2 surface, further preferred 0.20 mg to 1 mg and especially preferred 0.2 mg to 0.5 mg/cm2 surface.

Moreover preferred are embodiments, which contain an active agent in two layers. This can be two different active agents, too. If the same active agent is contained in two layers, it is preferred, that the two layers feature a different active agent concentration. Further it is preferred, when the lower layer features a smaller active agent concentration than the upper layer.

The stents according to invention can be manufactured by a method of biocompatible coating of stents, the principle of which is as follows:
a. Providing a stent, and
b. depositing at least one biostable polysulfone layer with or without at least one hydrophilic polymer, and
c. depositing and/or incorporating at least one antiproliferative, antiinflammatory, antiphlogistic and/or antithrombotic active agent on and/or in the biostable layer, or
b′. depositing at least one biostable polysulfone layer with or without the at least one hydrophilic polymer together with at least one antiproliferative, antiinflammatory, antiphlogistic and/or antithrombotic active agent.

After the step b′ preferably step c′ can follow:
c′. Depositing of at least one antiproliferative, antiinflammatory, antiphlogistic and/or antithrombotic active agent on the biostable polymer layer.

After the steps a, b and c or the steps a, b′ or the steps a, b′ and c′ still another step d can follow:
d. Depositing of at least a second biostable polysulfone layer.

After the steps a, b and c or the steps a, b′ or the steps a, b′ and c′ or the step d still another step d′ can follow:
d′. Depositing of at least one further layer of a biodegradable polymer.

The polysulfone layer of step d and/or the biodegradable layer of step d′ can contain an active agent (the same one or different active agents). The active agent can be different in each layer and can be present in the same or different concentrations. The polysulfone layer of step d can further comprise an hydrophilic polymer. If more than one polysulfone layers are present, each polysulfone layer can comprise the same or a different polysulfone and the same or a different hydrophilic polymer in the same or different concentration. Furthermore, each polysulfone layer can contain the same or a different active agent in the same or a different concentration.

In the preferred two layer embodiment of two polysulfone layers, the second biostable polysulfone layer can consist on the one hand of another polysulfone than the first subjacent layer and can contain on the other hand a different amount of the same or of another hydrophilic polymer. Preferred is, when this second biostable polysulfone layer contains at least one active agent. Especially preferred are embodiments with a biostable polysulfone layer with or without hydrophilic polymer as external layer.

The antiproliferative, antiinflammatory, antiphlogistic and/or antithrombotic active agent is preferably selected from the group listed above.

Further preferred are embodiments, which feature a hemocompatible layer. This hemocompatible layer consists of the above mentioned hemocompatible substances, especially of completely desulphated and N-reacetylated heparin, desulphated and N-reacetylated heparin, N-carboxymethylated, partially N-acetylated chitosan and/or of mixtures of these substances and is directly or indirectly deposited on the lower biostable layer. This hemocompatible layer can be located between two other layers as well as form the supreme layer. Embodiments with two hemocompatible layers are possible, too, but only one hemocompatible layer is preferred. The hemocompatible layer can be bound adhesively as well as covalently or partially adhesively and partially covalently to the subjacent layer.

The respective layers are deposited preferably via dipping or spraying method. Further the individual layers are preferably deposited on the subjacent layer only when that layer is in dry state.

Preferred is a method, which consists of the two steps a) and b′).

The coating principle offers a broad width of variation in respect of the proposed demands on the active agent and also on the properties of the used polysulfone, so that different variants of coating result, which can be also combined with each other.

The possibility to influence the properties of the polysulfone via the amount and molecular weight of the added hydrophilic polymer such as PVP represents in respect of the used active agents a broad field of flexibility of the components to a dovetailed system.

Further layers of polysulfone without addition of PVP and/or with equal or different PVP content with and without active agents are possible. Likewise a layer of completely N-deacetylated and reacetylated heparin, desulphated and N-reacetylated heparin, N-carboxymethylated and/or partially N-acetylated chitosan and/or of mixtures of these substances bound preferred covalently can be deposited directly on the stent surface. The athrombogeneous properties of this layer can mask the subjacent alien surface in case of damaging the adjacent biostable layer or layers as it arises, for example, in the preliminary stage or during the implantation by mechanical destruction of the coating. This inert layer can be used in case of need optionally covalently or adhesively between two layers and/or as top layer, too.

Variant A:

a.) Providing an uncoated stent,
b.) depositing one biostable polysulfone layer with or without hydrophilic polymer,
c.) depositing an active agent or active agent combination in and/or on the polysulfone layer via dipping or spraying method,
d.) substantially complete and/or incomplete coating of the biostable polysulfone layer containing the active agent with at least another biostable polysulfone layer corresponding to the first layer or differing from this first layer in its content of hydrophilic polymer and thereby in pore size,
e.) depositing the same or another active agent or active agent combination in and/or on the external biostable layer, so that different active agents and/or active agent combinations can be deposited on the stent in a targeted manner separated from each other by means of both layers, as well as in case of different pore size of the polymer a different active agent load can be realized as well as a different elution velocity of the same and/or another active agent is enabled.

The term “depositing” in step c) and/or step e) especially means “diffusion” of the active agent into the respective layer.

Preferred are medical products with two biostable polysulfone layers, which can contain different hydrophilic polymers in different concentrations.

The deposition of all provided polymer layers can be carried out before diffusion of the active agent in these layers, when the same active agent or active agent combination shall be contained in both layers.

Additionally another layer of a suitable polysulfone or of the pure hydrophilic polymer can be deposited as diffusion barrier and top coat.

Variant B

a.) Providing an uncoated stent,
b.) depositing one biostable polysulfone layer with or without hydrophilic polymer,
c.) substantially complete and/or incomplete coating of the biostable polysulfone layer with at least one antiproliferative, antiphlogistic and/or antithrombotic active agent and/or active agent combination via spraying method,
d.) substantially complete and/or incomplete coating of the active agent layer with at least another biostable polysulfone layer, which equals the first layer or differs from this first layer in its content of hydrophilic polymer and thereby in pore size, with or without active agent and/or active agent combination, and/or
d′.) substantially complete and/or incomplete coating of the active agent layer with a hydrophilic polymer as top coat with or without active agent and/or active agent combination.

With these variant one is able to fit the coating material to the active agents and also the temporally released amount of active agent to the requirements at the concerned segment.

In the case of multi layer systems the newly deposited layer substantially covers the subjacent layer completely. “Substantially” means by 50 to 100%, preferred 70 to 100%, further preferred 80 to 100%, further preferred 90 to 100% and especially preferred over 96% and especially further preferred over 98%.

In case of multi layer systems with two or more biostable layers, each biostable layer may contain a different amount of hydrophilic polymer. Furthermore, it is preferred in the case of multi layer systems that the at least one polysulfone layer with or without hydrophilic polymer is coated with a biodegradable polymer layer.

Also medical products with multi layer systems are preferred, especially with tow layers, wherein each layer contains a different concentration of an and/or a different kind of antiproliferative, antiinflammatory, antiphlogistic and/or antithrombotic active agent. The active agent can be adhesively or covalently bound to the respective layer.

Furthermore, it is preferred that the at least one polysulfone layer containing or not containing an active agent is coated or covered by a biodegradable layer containing no active agent or containing covalently and/or adhesively bound the same or a different active agent in the same or different concentration.

Object of the invention are also the medical products producible according to the aforementioned methods and especially stents.

The stents according to invention solve both the problem of acute thrombosis and the problem of neointima hyperplasia after a stent implantation. In addition the stents according to invention are especially well suitable due to their coating whether as single layer or as multi layer system for the continuous release of one or more anti proliferative, antiinflammatory, antiphlogistic, antithrombotic and/or immunosuppressive active agents. Due to this feature of aimed continuous active agent release in a required amount the coated stents according to invention prevent almost completely the danger of restenosis.

The prevention or reduction of restenosis takes place on the one hand by suppression of the cellular reactions during the first days and weeks after implantation by means of the selected active agents and active agent combinations and on the other hand by provision of a biocompatible surface, so that with decreasing influence of the active agent no reactions arise on the present alien surface, which would lead also to a restenosis of the blood vessel on a long term.

### DESCRIPTION OF THE FIGURES

FIG. 1: Elution diagram of macrocyclic carbon suboxide (MCS) in a triple layer system with polysulfone as base coating, the active agent as central layer and a polysulfone coating covering completely the central active agent layer with an amount of 0.04% of polyvinylpyrrolidone.

FIG. 2: Elution diagram of paclitaxel from a polysulfone matrix with an amount of 9.1% of polyvinylpyrrolidone.

FIG. 3: Elution diagram of simvastatin from pure polysulfone matrix without rate of hydrophilic polymer.

FIG. 4: Elution diagram of β-estradiol with a rate of 15% by weight in the pure polysulfone matrix without rate of hydrophilic polymer.

FIG. 5: Elution diagram of triazolopyrimidine (trapidil®) from a polysulfone matrix with an amount of 4.5% of polyvinylpyrrolidone.

FIG. 6: Elution diagram of triazolopyrimidine (trapidil®) with an amount of 50% in the pure polysulfone matrix.

FIG. 7: Photomicrography of the vessel segments after 4 weeks of implantation in pig.

FIG. 7A shows the cross-section through the segment of a matrix stent without active agent.

FIG. 7B shows a cross-section through the vessel segment with the polysulfone coated stent loaded in higher concentration with MCS.

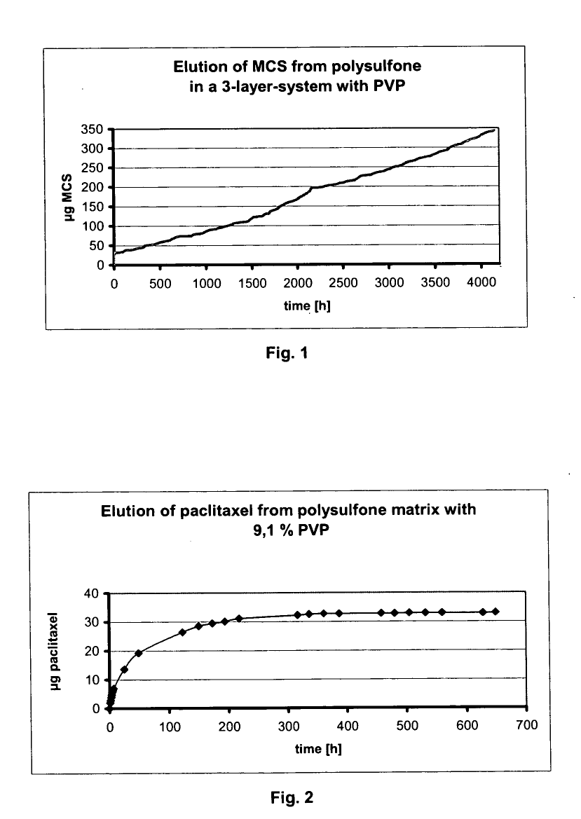

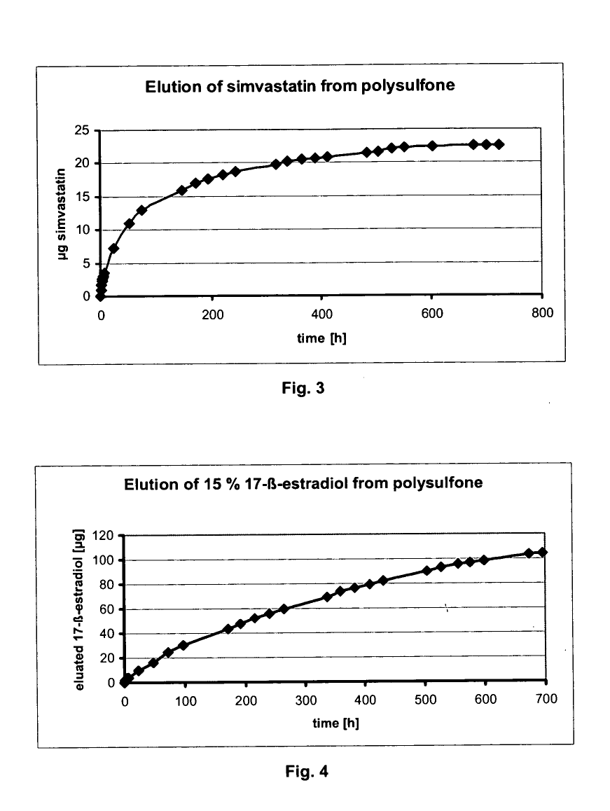

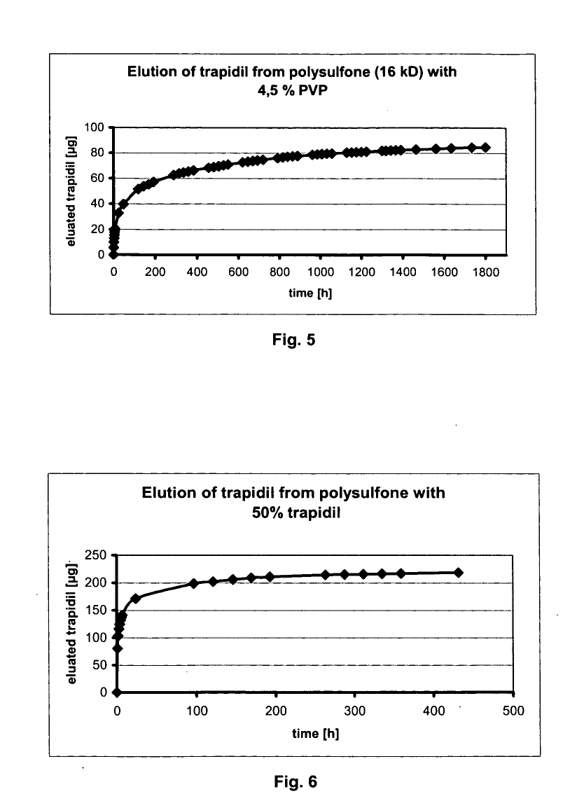

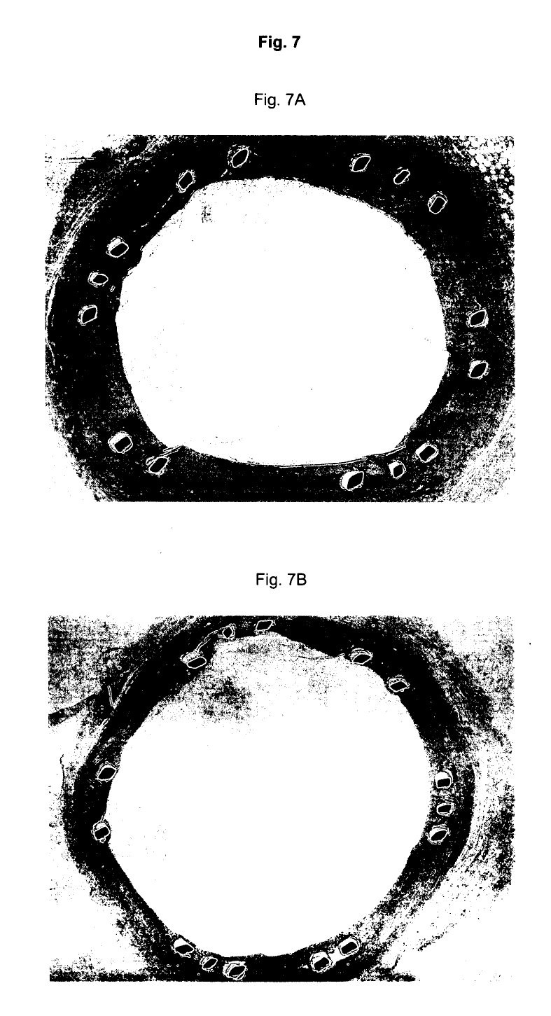

### EXAMPLES

### Example 1

Coating of Stents with Polyethersulfone

Spray solution:

| | |
|---|---|
| a. | PS solution: |
| | 176 mg of PS (polyethersulfone, Odel ®, purchasable |
| | at Solvay) are balanced |
| | and mixed with chloroform to 20 g. |
| | → 0.88% PS |

| | spray | before | after | |
|---|---|---|---|---|
| Stent | solution | coating | coating | coating mass |
| 1 | 2.0 ml | 0.01754 g | 0.01826 g | 0.72 mg |
| 2 | 2.0 ml | 0.01814 g | 0.01889 g | 0.75 mg |
| 3 | 2.0 ml | 0.01751 g | 0.01832 g | 0.81 mg |
| 4 | 2.0 ml | 0.01742 g | 0.01816 g | 0.74 mg |
| 5 | 2.0 ml | 0.01734 g | 0.01814 g | 0.80 mg |
| 6 | 2.0 ml | 0.01736 g | 0.01815 g | 0.80 mg |

### Example 2

Coating of Stents with Polyethersulfone (Base Coat) and Polyethersulfone with 0.04% PVP and. 0.08% PVP Resp. as Top Coat

Spray solutions:

| | |
|---|---|
| a. | Polysulfone solution: |
| | 17.6 mg of PS are balanced and mixed with chloroform to 2 g. |
| | → 0.88% PS |
| b. | Polysulfone/PVP solution: |
| | 25.2 mg of PS and 1.2 mg of PVP are balanced and mixed |
| | with chloroform to 3 g. |
| | → 0.84% PS, 0.04% PVP |
| b′. | Polysulfone/PVP solution: |
| | 24 mg of PS and 2.4 mg of PVP are balanced and |
| | mixed with chloroform to 3 g. |
| | → 0.80% PS, 0.08% PVP |

Spray coating:

The balanced stents are spray coated with the spray solutions in the
given sequence with a.) 0.5 ml and b.) 0.85 ml. Thereby after each spray
process a time period of at least 6 hours elapses until the next layer is
deposited. After drying at room temperature over night in the
clean room it is balanced again.

| Stent | before coating | after coating | coating mass |
|---|---|---|---|
| 1b | 0.02058 g | 0.02132 g | 0.75 mg |
| 1b′ | 0.01968 g | 0.02022 g | 0.54 mg |
| 2b′ | 0.01968 g | 0.02034 g | 0.66 mg |

### Example 3

Manufacture of Stents with MCS and Polyethersulfone in the 3-Layer-System According to Variant B

Spray solutions:

| | |
|---|---|
| a) | Polyethersulfone solution: (first layer: base coat): |
| | 70.4 mg of PS are balanced and mixed with chloroform to 8 g. |
| | → 0.88% PS |
| b) | MCS solution (2. layer: middle coat): |
| | 39.6 mg of MCS are balanced and mixed with 20% |
| | ethanol in water to 18 g. |
| | → 0.22% MCS |
| c) | Polyethersulfone/PVP solution (3. layer: top coat): |
| | 100.8 mg of PS and 4.8 mg of polyvinylpyrrolidone |
| | are balanced and mixed |
| | with chloroform to 12 g. |
| | → 0.84% PS, 0.04% PVP |

Spray coating:

Not expanded stainless steel stents are balanced and spray
coated after their cleaning. The stents are sprayed with the
corresponding amount of the respective spray solution with
a) 0.5 ml; b.) 1.5 ml and c.) 0.85 ml in the given sequence.
Thereby after each layer a time period of at least 6 hours
elapses until the next layer is sprayed. After drying at room
temperature over night it is balanced again. The average value
of the active agent content on the stents is 153 ± 9 μg.

| | before | after | | |
|---|---|---|---|---|
| Stent | coating | coating | coating mass | mass MCS |
| 1 | 0.01829 g | 0.01894 g | 0.65 mg | 141 μg |
| 2 | 0.01753 g | 0.01826 g | 0.73 mg | 159 μg |
| 3 | 0.01772 g | 0.01836 g | 0.64 mg | 139 μg |
| 4 | 0.01719 g | 0.01790 g | 0.71 mg | 154 μg |
| 5 | 0.01833 g | 0.01903 g | 0.70 mg | 152 μg |
| 6 | 0.01774 g | 0.01836 g | 0.62 mg | 135 μg |
| 7 | 0.01729 g | 0.01802 g | 0.73 mg | 159 μg |

### EXAMPLE 4

Determination of the Elution Kinetics of MCS from Polyethersulfone with 4.5% PVP

In each case one stent is given into a snap-on cap vial, mixed with 2 ml of PBS buffer, closed with parafilm and incubated for given times in the drying closet at 37° C. After elapsing of the chosen time period the supernatant is depipetted and its UV absorption at 207 nm is measured. The respective stent is again mixed with 2 ml of PBS and incubated again at 37° C. This process is repeated several times.

### Example 5

Coating of Stents with Simvastatin Loaded Polysulfone Matrix

Spray solutions:

| a. | PS/simvastatin solution: | | | | |
|---|---|---|---|---|---|
| | 26.4 mg of PS and 8.8 mg of simvastatin are balanced | | | | |
| | and mixed with chloroform to 4 g. | | | | |
| | → 0.66% PS, 0.22% simvastatin | | | | |
| b. | PS/simvastatin/PVP solution: | | | | |
| | 24.8 mg of PS, 8.8 mg of simvastatin und 1.6 mg of PVP | | | | |
| | are balanced and mixed with chloroform to 4 g. | | | | |
| | → 0.62% PS, 0.22% Simvastatin, 0.04% PVP | | | | |
| | spray | before | after | | mass |
| Stent | solution | coating | coating | coating mass | simvastatin |
| 1 | 2.0 ml a) | 0.02164 g | 0.02171 g | 1.08 mg | 270 μg |
| 2 | 2.0 ml b) | 0.02129 g | 0.02253 g | 1.24 mg | 310 μg |

### Example 6

Coating of Stents with Simvastatin Loaded Polysulfone Matrix with High PVP Rate

Spray solution:

| a. | PS/simvastatin/PVP solution: | | | | |
|---|---|---|---|---|---|
| | 23.2 mg of PS, 8.8 mg of simvastatin and 3.2 mg of PVP | | | | |
| | are balanced and mixed with chloroform to 4 g. | | | | |
| | → 0.58% PS, 0.22% simvastatin, 0.08% PVP | | | | |
| | spray | before | after | | mass |
| Stent | solution | coating | coating | coating mass | simvastatin |
| 1 | 2.0 ml a) | 0.02164 g | 0.02171 g | 1.08 mg | 270 μg |
| 2 | 2.0 ml a) | 0.02129 g | 0.02253 g | 1.24 mg | 310 μg |

### Example 7

Coating of Stents with Paclitaxel Loaded Polysulfone Matrix Matrix

Spray solutions:

| a. | PS/paclitaxel solution: | | | | |
|---|---|---|---|---|---|
| | 13.2 mg of PS and 4.4 mg of paclitaxel are balanced and | | | | |
| | mixed with chloroform to 2 g. | | | | |
| | → 0.66% PS, 0.22% paclitaxel | | | | |
| b. | PS/PVP/paclitaxel solution: | | | | |
| | 11.6 mg of PS, 1.6 mg of PVP and 4.4 mg of paclitaxel are | | | | |
| | balanced and mixed with chloroform to 2 g. | | | | |
| | → 0.58% PS, 0.08% PVP, 0.22% paclitaxel | | | | |
| | spray | before | after | | mass |
| Stent | solution | coating | coating | coating mass | paclitaxel |
| 1 | 1.0 ml a) | 0.01725 g | 0.01770 g | 0.45 mg | 113 μg |
| 2 | 1.0 ml b) | 0.01735 g | 0.01790 g | 0.55 mg | 138 μg |

### Example 8

Coating of Stents with 17-β-estradiol in Polysulfone Matrix

Spray solutions:

| | |
|---|---|
| a. | PS/25% 17-β-estradiol solution: |
| | 46.2 mg of PS and 15.4 mg of 17-β-estradiol are balanced |
| | and mixed with chloroform to 7 g. |
| | → 0.66% PS, 0.22% 17-β-estradiol |
| b. | PS/20% 17-β-estradiol solution: |
| | 28.2 mg of PS and 7 mg of 17-β-estradiol are balanced |
| | and mixed with chloroform to 4 g. |
| | → 0.704% PS, 0.176% 17-β-estradiol |
| c. | PS/15% 17-β-estradiol solution: |
| | 29.9 mg of PS and 5.3 mg of 17-β-estradiol are balanced |
| | and mixed with chloroform to 4 g. |
| | → 0.748% PS, 0.132% 17-β-estradiol |

| | spray | before | after | | mass |
|---|---|---|---|---|---|
| Stent | solution | coating | coating | coating mass | 17-β-estradiol |
| 1 | 2.2 ml a) | 0.02052 g | 0.02166 g | 1.14 mg | 285 μg |
| 2 | 2.2 ml a) | 0.02065 g | 0.02189 g | 1.24 mg | 310 μg |
| 3 | 2.2 ml b) | 0.02080 g | 0.02206 g | 1.27 mg | 254 μg |
| 4 | 2.2 ml c) | 0.02064 g | 0.02213 g | 1.49 mg | 224 μg |

### Example 9

Coating of Stents with a Triazolopyrimidine (Trapidil®) Containing Polysulfone Matrix

Spray solution:

| | PS/trapidil solution: | | | | |
|---|---|---|---|---|---|
| | 19.8 mg of PS and 6.6 mg of trapidil ® are balanced | | | | |
| | and mixed with chloroform to 3 g. | | | | |
| | → 0.66% PS, 0.22% trapidil ® | | | | |
| | spray | before | after | | mass |
| Stent | solution | coating | coating | coating mass | trapidil ® |
| 1 | 1.7 ml | 0.01742 g | 0.01855 g | 1.13 mg | 283 μg |

### Example 10

In Vivo Experiments of Stents with Polyethersulfone as Matrix with and without Macrocyclic Suboxide

Polyethersulfone coated stents were implanted into the coronary arteries of 13 domestic pigs of different sex with 20-25 kg of weight. Three groups of stents were distinguished. One group contained a high dosage of paclitaxel, the second contained a low dosage of paclitaxel and the last group was the pure matrix stent without active agent additive. After four weeks the stents were removed and analyzed for inflammation reactions (peri-strut) and neointima formation.

Histomorphometric evaluation after 4 weeks of implantation time

| | | intima | | |
|---|---|---|---|---|
| | amount | thickness | | grade of |
| Coating | of stents | [mm] | stenosis [%] | injury |
| Matrix/high active | 6 | 0.14 ± 0.06 | 19 ± 9 | 0.32 ± 0.19 |
| agent concentration | | | | |
| Matrix/low active | 6 | 0.23 ± 0.07 | 32 ± 10 | 0.46 ± 0.29 |
| agent concentration | | | | |
| Matrix without | 4 | 0.17 ± 0.06 | 23 ± 8 | 0.15 ± 0.12 |
| active agent | | | | |

All analyzed stents showed independently of the coating only minimal inflammations around the stent struts and on the adventitia. The in average higher intima thickness of the stents with the low active agent load could be attributed to the stronger overexpansion of the vessel during the implantation. The pure matrix stent shows no conspicuities to be assigned to the polymer in the vessel reactions, what emphasizes its hemocompatibility and suitability as active agent substrate.

### Example 11

In Vivo Experiments of Stents with Polyethersulfone as Matrix with and without Paclitaxel

Analogously to the previous Example 10 polyethersulfone coated stents were compared to paclitaxel loaded polyethersulfone coated stents:

Histomorphometric evaluation after 4 weeks of implantation time

| | amount | intima | | grade of |
|---|---|---|---|---|
| Coating | of stents | thickness [mm] | stenosis [%] | injury |
| Matrix/active | 6 | 0.21 ± 0.10 | 26 ± 12 | 0.23 ± 0.20 |
| agent | | | | |
| Matrix | 4 | 0.14 ± 0.06 | 18 ± 8 | 0.10 ± 0.07 |

The results of this study show the profit of the polysulfone coating.

### Example 12

Preparation of the Polysulfone According to Formula (IIA).

The polysulfone (IIA) was prepared according to the instruction of E. Avram et al. J. Macromol Sci. Pure Appl. Chem., 1997, A34, 1701. 3 mole equivalents of benzyl alcohol are dissolved in toluene and deprotonated with sodium. 1 mole equivalent of the polysulfone (IIA) is added and afterwards the reaction mixture is heated to boiling temperature. The reaction product is obtained in a yield of 22%.

### Example 13

Preparation of the Polysulfone According to Formula (IIC):

The polysulfone (IIC) was prepared according to the instruction of M. D. Guiver et al., Brit. Polym. L. 1990, 23, 29.

1 g of the obtained polysulfone (IIC) was ersterified by using ortho ethyl acetate, whereas toluene was used as solvent and the volatile reaction products were removed from the reaction equilibrium via distillation. 40% of the carboxylate groups were converted into ethylester groups. According to example 7 this polymer was deposited together with paclitaxel on a stent. The stent shows good hemocompatibility and an amorphous polysulfone coating, which was suitable for the controlled release of paclitaxel.

### Example 14

1 g of the prepared polysulfone according to example 12 is admixed with 200 mg of the polysulfone according to formula (IIC) and deposited according to example 7 together with the active agent paclitaxel on a stent. The coated stent features a good hemocompatibility and an amorphous polysulfone coating, which was suitable for the controlled release of paclitaxel.

### Example 15

Introduction of Chlorosulfone Groups in Polysulfone.

2.4 g of polysulfone is dissolved in 700 ml of chloroform and cooled to −20° C. Afterwards 23.3 ml of chlorosulfonic acid are slowly added dropwise. As the reaction is strongly exothermic the reaction vessel is cooled in the ice bath. After adding the chlorosulfonic acid the solution is let to heat up to room temperature under stirring. After 30 minutes the polymer is precipitated in ethanol and afterwards rinsed with deionized water. To remove the chlorosulfonic acid completely it is extracted again for 10 minutes in deionized water.

### Example 16

S-alkoxy-de-chloration.

10 g of ethanol are mixed with 100 ml of water and admixed with 2-3 drops of methyl red in acetone. This solution is given on 5 g of fine granulate chlorosulfonated polysulfone. The solution is added drop wise with 5N KOH until the change of color from yellow to red occurs. Afterwards the vessel is closed and shaken well. Potassium hydroxide solution is added and shaken until the change of color fails to appear. The formed polysulfone ester is filtered, washed with water and recrystallized for purification.

### Example 17

S-alkoxy-de-chloration.

10 g of dry ethanol are mixed with 60 ml of pyridine. This solution is added under ice cooling to 40 g of fine pulverized chlorosulfonated polysulfone. Afterwards it is stirred under exclusion of moisture over night at room temperature. Afterwards the suspension is added into iced water and acidified carefully with concentrated hydrochloric acid. The washing is carried out with aqueous hydrogen carbonate solution. After filtration the esterified polysulfone can be recrystallized.

### Example 18

Coating with a Mixture of Polysulfone and Polysulfone According to Formula (IIC).

24 mg PS and 2.4 mg of polysulfone according to formula (IIC) are balanced and mixed with chloroform to 3 g.

→ 0.80% PS, 0.08% PVP

A stent is coated according to example 7 with this mixture by the spraying method.

1. Medical product, characterized in that its surface is covered at least in part with at least one biostable polysulfone layer.

2. Medical product according to claim 1, characterized in that the polysulfone is selected from the group comprising: polyethersulfone, substituted polyethersulfone, polyphenylsulfone, substituted polyphenylsulfone, polysulfone block copolymers, perfluorinated polysulfone block copolymers, semifluorinated polysulfone block copolymers, substituted polysulfone block copolymers and/or mixtures of the aforementioned polymers.

3. Medical product according to claim 1, characterized in that the at least one biostable polysulfone layer comprises at least one hydrophilic polymer.

4. Medical product according to claim 3, characterized in that the polysulfone with the at least one hydrophilic polymer is present in a mixture ratio of 50% by weight:50% by weight up to 99.999% by weight:0.001% by weight.

5. Medical product according to claim 3, characterized in that the hydrophilic polymer is selected from the group comprising: polyvinylpyrrolidone, glycerine, polyethylene glycol, polypropylene glycol, polyvinyl alcohol, polyhydroxyethyl methacrylates, polyacrylamide, polyvalerolactones, poly-ε-decalactones, polylactone acid, polyglycolic acid, polylactides, polyglycolides, copolymers of polylactides and polyglycolides, poly-ε-caprolactone, polyhydroxybutanoic acid, polyhydroxybutyrates, polyhydroxyvalerates, polyhydroxybutyrate-co-valerates, poly(1,4-dioxane-2,3-diones), poly(1,3-dioxane-2-ones), poly-p-dioxanones, polyanhydrides such as polymaleic anhydrides, fibrin, polycyanoacrylates, polycaprolactoned imethylacrylates, poly-b-maleic acid, polycaprolactone butylacrylates, multiblock polymers from oligocaprolactone dioles and oligodioxanone dioles, polyether ester multiblock polymers from PEG and polybutylene terephthalate, polypivotolactones, polyglycolic acid trimethyl-carbonates, polycaprolactone-glycolides, poly-g-ethylglutamate, poly(DTH-iminocarbonate), poly(DTE-co-DT-carbonate), poly(bisphenol-A-iminocarbonate), polyorthoesters, polyglycolic acid trimethyl-carbonates, polytrimethylcarbonates, polyiminocarbonates, poly(N-vinyl)-pyrrolidone, polyvinylalcohols, polyesteramides, glycolated polyesters, polyphosphoesters, polyphosphazenes, poly[p-carboxyphenoxy)propane], polyhydroxypentanoic acid, polyanhyd rides, polyethyleneoxide-propyleneoxide, soft polyurethanes, polyurethanes with amino acid residues in the backbone, polyether esters, polyethyleneoxide, polyalkeneoxalates, polyorthoesters as well as copolymers thereof, lipids, carrageenans, fibrinogen, starch, collagen, protein based polymers, polyamino acids, synthetic polyamino acids, zein, modified zein, polyhydroxyalkanoates, pectic acid, actinic acid, modified and non modified fibrin and casein, carboxymethyl sulphate, albumin, hyaluronic acid, chitosan and its derivatives, chondroitine sulphate, dextran, b-cyclodextrins, copolymers with PEG and polypropylene glycol, gum arabicum, guar, gelatine, collagen, collagen-N-hydroxysuccinimide, lipids, phospholipids, modifications and copolymers and/or mixtures of the afore mentioned substances.

6. Medical product according to claim 5, characterized in that the hydrophilic polymer is selected from the group comprising: polyvinylpyrrolidone polyethylene glycol, polypropylene glycol and/or glycerine.

7. Medical product according to claim 1, characterized in that a pore size of the polysulfone coating is determined by the mixing ratio of polysulfone with the at least one hydrophilic polymer.

8. Medical product according to claim 1, characterized in that at least one antiproliferative, antiinflammatory, antiphlogistic and/or antithrombotic active agent is present in, under and/or on the at least one biostable polysulfone layer with or without the at least one hydrophilic polymer.

9. Medical product according to claim 1, characterized in that the biostable layer is bound adhesively or covalently on the surface of the medical product.

10. Medical product according to claim 1, characterized in that the coating of the surface of the medical product consists of one, two, three or more layers.

11. Medical product according to claim 1, characterized in that at least one layer of completely desulphated and N-reacetylated heparin, desulphated and N-reacetylated heparin, N-carboxymethylated and/or partially N-acetylated chitosan and/or mixtures of these substances is present under and/or on the at least one biostable polysulfone layer with or without the at least one hydrophilic polymer.

12. Medical product according to claim 1, characterized in that the at least one antiproliferative, antiinflammatory, antiphlogistic and/or antithrombotic active agent is selected from the group comprising: sirolimus (rapamycin), everolimus, somatostatin, tacrolimus, roxithromycin, dunaimycin, ascomycin, bafilomycin, erythromycin, midecamycin, josamycin, concanamycin, clarithromycin, troleandomycin, folimycin, cerivastatin, simvastatin, lovastatin, fluvastatin, rosuvastatin, atorvastatin, pravastatin, pitavastatin, vinblastine, vincristine, vindesine, vinorelbine, etoposide, teniposide, nimustine, carmustine, lomustine, cyclophosphamide, C-type natriuretic peptide (CNP), 4-hydroxycyclophosphamide, estramustine, melphalan, ifosfamide, trofosfamide, chlorambucil, bendamustine, dacarbazine, busulfan, procarbazine, treosulfan, temozolomide, thiotepa, daunorubicin, doxorubicin, aclarubicin, epirubicin, mitoxantrone, idarubicin, bleomycin, mitomycin, dactinomycin, methotrexate, fludarabine, fludarabine-5′-dihydrogenphosphate, cladribine, mercaptopurine, thioguanine, cytarabine, fluorouracil, gemcitabine, capecitabine, docetaxel, carboplatin, cisplatin, oxaliplatin, amsacrine, irinotecan, topotecan, hydroxycarbamide, miltefosine, pentostatin, aldesleukin, tretinoin, asparaginase, pegaspargase, anastrozole, exemestane, letrozole, formestane, aminoglutethimide, adriamycin, azithromycin, spiramycin, cepharantin, smc proliferation inhibitor-2w, epothilone A and B, mitoxantrone, azathioprine, mycophenolatmofetil, c-myc-antisense, b-myc-antisense, betulinic acid, camptothecin, lapachol, 1-lapachone, podophyllotoxin, betulin, podophyllic acid 2-ethylhydrazide, molgramostim (rhuGM-CSF), peginterferon α-2b, lenograstim (r-HuG-CSF), filgrastim, macrogol, dacarbazine, basiliximab, daclizumab, selectin (cytokine antagonist), CETP inhibitor, cadherines, cytokinin inhibitors, COX-2 inhibitor, NFkB, angiopeptin, ciprofloxacin, camptothecin, fluroblastin, monoclonal antibodies, which inhibit the muscle cell proliferation, bFGF antagonists, probucol, prostaglandins, 1,11-dimethoxycanthin-6-one, 1-hydroxy-11-methoxycanthin-6-one, scopoletin, colchicine, NO donors such as pentaerythritol tetranitrate and syndnoeimines, S-nitrosoderivatives, tamoxifen, staurosporine, β-estradiol, α-estradiol, estrone, estriol, ethinylestradiol, fosfestrol, medroxyprogesterone, estradiol cypionates, estradiol benzoates, tranilast, kamebakaurin and other terpenoids, which are applied in the therapy of cancer, verapamil, tyrosine kinase inhibitors (tyrphostines), cyclosporine A, paclitaxel and derivatives thereof such as 6-α-hydroxy-paclitaxel, baccatin, taxotere and other, synthetically produced as well as from native sources obtained macrocyclic oligomers of carbon suboxide (MCS) and derivatives thereof, mofebutazone, acemetacin, diclofenac, lonazolac, dapsone, o-carbamoylphenoxyacetic acid, lidocaine, ketoprofen, mefenamic acid, piroxicam, meloxicam, chloroquine phosphate, penicillamine, hydroxychloroquine, auranofin, sodium aurothiomalate, oxaceprol, celecoxib, β-sitosterin, ademetionine, myrtecaine, polidocanol, nonivamide, levomenthol, benzocaine, aescin, ellipticine, D-24851 (Calbiochem), colcemid, cytochalasin A-E, indanocine, nocodazole, S 100 protein, bacitracin, vitronectin receptor antagonists, azelastine, guanidyl cyclase stimulator, tissue inhibitor of metal proteinase-1 and -2, free nucleic acids, nucleic acids incorporated into virus transmitters, DNA and RNA fragments, plasminogen activator inhibitor-1, plasminogen activator inhibitor-2, antisense oligonucleotides, VEGF inhibitors, IGF-1, active agents from the group of antibiotics such as cefadroxil, cefazolin, cefaclor, cefotaxim, tobramycin, gentamycin, penicillins such as dicloxacillin, oxacillin, sulfonamides, metronidazol, antithrombotics such as argatroban, aspirin, abciximab, synthetic antithrombin, bivalirudin, coumadin, enoxaparin, desulphated and N-reacetylated heparin (hemoparin®), tissue plasminogen activator, GpIIb/IIIa platelet membrane receptor, factor Xₐ inhibitor antibody, heparin, hirudin, r-hirudin, PPACK, protamin, prourokinase, streptokinase, warfarin, urokinase, vasodilators such as dipyramidole, triazolopyrimidine (trapidil®), nitroprussides, PDGF antagonists such as triazolopyrimidine and seramin, ACE inhibitors such as captopril, cilazapril, lisinopril, enalapril, losartan, thiolprotease inhibitors, prostacyclin, vapiprost, interferon α, β and γ, histamine antagonists, serotonin blockers, apoptosis inhibitors, apoptosis regulators such as p65 NF-kB or Bcl-xL antisense oligonucleotides, halofuginone, nifedipine, tocopherol, tranilast, molsidomine, tea polyphenols, epicatechin gallate, epigallocatechin gallate, Boswellic acids and derivatives thereof, leflunomide, anakinra, etanercept, sulfasalazine, etoposide, dicloxacillin, tetracycline, triamcinolone, mutamycin, procainamid, retinoic acid, quinidine, disopyramide, flecainide, propafenone, sotalol, amidorone, natural and synthetically produced steroids such as bryophyllin A, inotodiol, maquiroside A, ghalakinoside, mansonine, strebloside, hydrocortisone, betamethasone, dexamethasone, non-steroidal substances (NSAIDS) such as fenoprofen, ibuprofen, indomethacin, naproxen, phenylbutazone and other antiviral agents such as acyclovir, ganciclovir and zidovudine, antimycotics such as clotrimazole, flucytosine, griseofulvin, ketoconazole, miconazole, nystatin, terbinafine, antiprozoal agents such as chloroquine, mefloquine, quinine, moreover natural terpenoids such as hippocaesculin, barringtogenol-C21-angelate, 14-dehydroagrostistachin, agroskerin, agrostistachin, 17-hydroxyagrostistachin, ovatodiolids, 4,7-oxycycloanisomelic acid, baccharinoids B1, B2, B3 and B7, tubeimoside, bruceanol A, B and C, bruceantinoside C, yadanziosides N and P, isodeoxyelephantopin, tomenphantopin A and B, coronarin A, B, C and D, ursolic acid, hyptatic acid A, zeorin, iso-iridogermanal, maytenfoliol, effusantin A, excisanin A and B, longikaurin B, sculponeatin C, kamebaunin, leukamenin A and B, 13,18-dehydro-6-α-senecioyloxychaparrin, taxamairin A and B, regenilol, triptolide, moreover cymarin, apocymarin, aristolochic acid, anopterin, hydroxyanopterin, anemonin, protoanemonin, berberine, cheliburin chloride, cictoxin, sinococuline, bombrestatin A and B, cudraisoflavone A, curcumin, dihydronitidine, nitidine chloride, 12-β-hydroxypregnadiene-4,16-diene-3,20-dione, bilobol, ginkgol, ginkgolic acid, helenalin, indicine, indicine-N-oxide, lasiocarpine, inotodiol, glycoside 1a, podophyllotoxin, justicidin A and B, larreatin, malloterin, mallotochromanol, isobutyrylmallotochromanol, maquiroside A, marchantin A, maytansine, lycoridicin, margetine, pancratistatin, liriodenine, bisparthenolidine, oxoushinsunine, aristolactam-AII, periplocoside A, bisparthenolidine, periplocoside A, ghalakinoside, ursolic acid, deoxypsorospermin, psychorubin, ricin A, sanguinarine, manwu wheat acid, methylsorbifolin, sphatheliachromen, stizophyllin, mansonine, strebloside, akagerine, dihydrousambarensine, hydroxyusambarine, strychnopentamine, strychnophylline, usambarine, usambarensine, berberine, liriodenine, oxoushinsunine, daphnoretin, lariciresinol, methoxylariciresinol, syringaresinol, umbelliferon, afromoson, acetylvismione B, desacetylvismione A, vismione A and B.

13. Medical product according to claim 11, characterized in that the at least one antiproliferative, antiinflammatoric, antiphlogistic and/or antithrombotic active agent is selected from the group comprising: paclitaxel and its derivatives, β-estradiol, simvastatin, PI-88 (sulphated oligosaccharide; Progen Ind.), macrocyclic carbon suboxides (MCS) and their derivatives, trapidil®, N-(pyridine-4-yl)-[1-4-(4-chlorobenzyl)-indol-3-yl]-glyoxylamide (D-24851), activated protein C (aPC), Ac-YVAD-CMK, Anginex (β-Pep25), Neovastat®, cryptophycin 52, and tacrolimus.

14. Medical product according to claim 1, characterized in that the at least one antiproliferative, antiinflammatoric, antiphlogistic and/or antithrombotic active agent is contained in a pharmaceutically active concentration of 0.001 to 20 mg per cm² of surface.

15. Medical product according to claim 1, characterized in that in the case of multiple layer systems the last layer is a pure active agent layer covalently and/or adhesively bound.

16. Method of biocompatible coating of medical products, characterized in the steps:
a. Providing a stent, and
b. depositing at least one biostable polysulfone layer with or without at least one hydrophilic polymer, and
c. depositing and/or incorporating at least one antiproliferative, antiinflammatory, antiphlogistic and/or antithrombotic active agent on and/or in the biostable layer, or
b′. depositing at least one biostable polysulfone layer with or without the at least one hydrophilic polymer together with at least one antiproliferative, antiinflammatory, antiphlogistic and/or antithrombotic active agent.

17. Method according to claim 16, comprising the step b′ and the further step:
c′. Depositing of at least one antiproliferative, antiinflammatory, antiphlogistic and/or antithrombotic active agent on the biostable polymer layer.

18. Method according to claim 16, comprising the further step:
d. Depositing of at least a second biostable polysulfone layer.

19. Method according to claim 16, characterized in that on and/or under the at least one biostable polysulfone layer at least one layer of completely desulphated and N-reacetylated heparin, desulphated and N-reacetylated heparin, N-carboxymethylated and/or partially N-acetylated chitosan and/or of mixtures of these substances is deposited.

20. Medical products obtainable accordingly to one method according to claim 16.

21. Medical products according to claim 1, characterized in that the at least one antiproliferative, antiinflammatory, antiphlogistic and/or antithrombotic active agent is released in a controlled manner through the surface coating.

22. Medical products according to claim 21, characterized in that the respective antiproliferative, antiinflammatory, antiphlogistic and/or antithrombotic active agent is contained in a pharmaceutically active concentration of 0.001-10 mg per cm² of medical product surface and per layer bearing the active agent.

23. Medical products according to claim 1, characterized in that in respect of the medical product a stent is concerned.
